(19) United States
(12) Patent Application Publication
(10) Pub. No.: US 2021/0371936 A1
(43) Pub. Date: Dec. 2, 2021

(54) PREDICTIVE METHODS IN BREAST CANCER

(71) Applicants: BioNTech Diagnostics GmbH, Mainz (DE); Stratifyer Molecular Pathology GmbH, Koln (DE)

(72) Inventors: Mark Laible, Mainz (DE); Michael Oed, Mainz (DE); Ralph Wirtz, Mainz (DE)

(21) Appl. No.: 17/291,224

(22) PCT Filed: Oct. 23, 2019
(86) PCT No.: PCT/EP2019/078883

§ 371 Date: May 4, 2021

(30) Foreign Application Priority Data
Nov. 5, 2018 (EP) PCT/EP2018/080161

(51) Int. Cl. C12Q 1/6886; G16B 20/00

(52) U.S. Cl. CPC C12Q 1/6886; C12Q 2600/118; C12Q 2600/158; G16B 20/00

(57) ABSTRACT

The present invention relates to methods of predicting the probability of an Oncotype DX® low risk recurrence score (RS) result (RS≤25) for a breast cancer patient, to methods for selecting a breast cancer treatment, and to methods of treatment of breast cancer. It also relates to the use of a kit in these methods.

### TECHNICAL FIELD OF THE INVENTION

The present invention relates to methods of predicting the probability of an Oncotype DX® low risk recurrence score (RS) result (RS≤25) for a breast cancer patient, to methods for selecting a breast cancer treatment, and to methods of treatment of breast cancer. It also relates to the use of a kit in these methods.

### BACKGROUND OF THE INVENTION

Biomarkers in early-stage breast cancers are essential for prognostic purposes and for guiding therapeutic decisions. Estrogen receptor (ESR1/ER), progesterone receptor (PGR/PR) and human epidermal growth factor receptor 2 (ERBB2/HER2), the main biomarkers used in the pathological workup of breast carcinomas, are routinely assessed by immunohistochemistry (IHC) and in equivocal cases by in-situ hybridization (ISH) (Goldhirsch et al., 2013). While the prognostic and predictive information that can be obtained by the proliferation marker Ki-67 (MKI67) are undisputed (Yerushalmi et al., 2010), routine application of this marker suffers from insufficient reproducibility (Varga et al., 2012; Polley et al., 2013).

In recent years, the panel of prognostic and predictive tools in breast cancer diagnosis has substantially expanded due to the incorporation of multigene assays, mainly for ER-positive/HER2-negative carcinomas. The growing acceptance of these tests as supplementary argument in chemotherapy decisions in early-stage breast cancer has been propelled by the increasing number of validation studies showing that these tests can indeed predict the response to chemotherapy—at least in large patient cohorts (Levine et al., 2016; Martin et al., 2015; Harris et al., 2016). Among these assays, the Oncotype DX® recurrence score (RS) is one of the most extensively studied recurrence risk classifiers that has been validated in a prospective setting (Sparano et al., 2015). Unfortunately, it is also one of the most expensive assays, remaining beyond reach for a large number of breast cancer patients.

Several investigators have studied whether traditional histological and immunohistochemical parameters can predict RS and could, thus, serve as cost-effective substitutes for the test, filtering out those patients with low- or high-risk tumors that could be spared further costly tests. Scoring algorithms have been presented that can predict the RS based on conventional histopathological prognosticators (Flanagan et al., 2008; Klein et al., 2013; Ingoldsby et al., 2013; Sahebjam et al., 2011; Turner et al., 2015; Harowicz et al., 2017; Kim et al., 2016). Most of these algorithms are based on semi-quantitative IHC and hence lack standardization across different laboratories, especially for the proliferation marker Ki-67. Yet, additional levels of analytical standardization can be obtained, as recently highlighted in a study investigating different quantification methodologies (Bartlett et al., 2016).

Recent work (Sparano et al., 2018) showed that, in a prospective clinical trial, an RS≤25 indicated a lack of benefit from chemotherapy (compared to endocrine therapy only) in postmenopausal women older than 50 years. A test which can be applied locally, with short turnaround times and at a low cost which would enable a safe prediction of an RS≤25 result would, thus, for a considerable proportion of patients reduce the waiting time for testing results and at the same time provide access for patients to high quality molecular gene expression diagnostics which are currently not in the reach for many patients due to high costs and lack of reimbursement.

Accordingly, it was an object of the present invention to provide an affordable and locally performed predictor of the Oncotype DX® RS (Varga et al., 2017), which can be applied in a highly standardized and reliable manner.

This and other objects are solved by the present invention, which will be described in the following.

### SUMMARY OF THE INVENTION

In one aspect, the present invention relates to a method of predicting the probability of an Oncotype DX® low risk recurrence score (RS) result (RS≤25) for an ERBB2-negative breast cancer patient, said method comprising:
calculating a score unscaled (su) based on the relative expression levels of mRNA of ESR1, PGR and MKI67 in a breast tumor sample of the breast cancer patient as determined by reverse transcription quantitative PCR (RT-qPCR), wherein
a) a higher score su indicates a higher probability of RS≤25, wherein a higher relative expression level of mRNA of ESR1 is associated with a higher su, a higher relative expression level of mRNA of PGR is associated with a higher su, and a higher relative expression level of mRNA of MKI67 is associated with a lower su; or
b) a lower score su indicates a higher probability of RS≤25, wherein a higher relative expression level of mRNA of ESR1 is associated with a lower su, a higher relative expression level of mRNA of PGR is associated with a lower su, and a higher relative expression level of mRNA of MKI67 is associated with a higher su.

In one embodiment, the method comprises, prior to calculating su:
determining the relative expression levels of mRNA of ESR1, PGR and MKI67 in the breast tumor sample by RT-qPCR.

In one embodiment, the breast cancer is an ERBB2-negative and ESR1-positive breast cancer.

In one embodiment, in the calculation of su, the relative expression levels (RELs) of mRNA of ESR1, PGR and MKI67 are weighted as follows:

REL(ESR1):REL(PGR):REL(MKI67)=0.60(±0.09):1(±0.15):1.78(±0.27).

In one embodiment, a higher score su indicates a higher probability of RS≤25, wherein su is calculated by using the formula:

su=BASELINE+WF(ESR1)·REL(ESR1)+WF(PGR)·REL(PGR)−WF(MKI67)·REL(MKI67),

wherein WF(ESR1) is a weighting factor for REL(ESR1), WF(PGR) is a weighting factor for REL(PGR), and WF(MKI67) is a weighting factor for REL(MKI67).

In one embodiment, a higher score su indicates a higher probability of RS≤25, wherein su is calculated by using the formula:

su=12.313+0.539·REL(ESR1)+0.902·REL(PGR)−1.602·REL(MKI67).

In one embodiment, a lower score su indicates a higher probability of RS≤25, wherein su is calculated by using the formula:

su=−BASELINE−WF(ESR1)·REL(ESR1)−WF(PGR)·REL(PGR)+WF(MKI67)·REL(MKI67),

wherein WF(ESR1) is a weighting factor for REL(ESR1), WF(PGR) is a weighting factor for REL(PGR), and WF(MKI67) is a weighting factor for REL(MKI67).

In one embodiment, a lower score su indicates a higher probability of RS≤25, wherein su is calculated by using the formula:

su=−12.313−0.539·REL(ESR1)−0.902·REL(PGR)+1.602·REL(MKI67).

In one embodiment, the method further comprises:
calculating a predicted probability of RS≤25 q, wherein
a) if a higher score su indicates a higher probability of RS≤25, q is calculated by using the formula

$$q = \frac{exp ( s u )}{( 1 + exp ( s u ) )} ;$$

and
b) if a lower score su indicates a higher probability of RS≤25, q is calculated by using the formula

$$q = 1 - \frac{exp ( s u )}{( 1 + exp ( s u ) )} ,$$

wherein, preferably, a q which is equal to or greater than a pre-defined threshold indicates a high probability of RS≤25, and a q which is lower than a pre-defined threshold indicates a low probability of RS≤25.

In one embodiment, the method further comprises:
calculating a clinical score s based on su, wherein s has a scale from 0 to 100 or from −10 to 10.

In one embodiment,
a) if a higher score su indicates a higher probability of RS≤25, a score s or a score su which is equal to or greater than a pre-defined threshold indicates a high probability of RS≤25, and a score s or a score su which is lower than the pre-defined threshold indicates a low probability of RS≤25; and
b) if a lower score su indicates a higher probability of RS≤25, a score s or a score su which is lower than a pre-defined threshold indicates a high probability of RS≤25, and a score s or a score su which is equal to or greater than the pre-defined threshold indicates a low probability of RS≤25.

In another aspect, the present invention relates to a method of predicting the probability of an Oncotype DX® low risk recurrence score (RS) result (RS≤25) for an ERBB2-negative breast cancer patient, said method comprising:
calculating a score unscaled (su) based on the relative expression levels of mRNA of PGR and MKI67 in a breast tumor sample of the breast cancer patient as determined by reverse transcription quantitative PCR (RT-qPCR), wherein
a) a higher score su indicates a higher probability of RS≤25, wherein a higher relative expression level of mRNA of PGR is associated with a higher su, and a higher relative expression level of mRNA of MKI67 is associated with a lower su; or
b) a lower score su indicates a higher probability of RS≤25, wherein a higher relative expression level of mRNA of PGR is associated with a lower su, and a higher relative expression level of mRNA of MKI67 is associated with a higher su.

In one embodiment, the method comprises, prior to calculating su:
determining the relative expression levels of mRNA of PGR and MKI67 in the breast tumor sample by RT-qPCR.

In one embodiment, the breast cancer is an ERBB2-negative and ESR1-positive breast cancer.

In one embodiment, in the calculation of su, the relative expression levels (RELs) of mRNA of PGR and MKI67 are weighted as follows:

REL(PGR):REL(MKI67)=1(±0.15):1.60(±0.24).

In one embodiment, a higher score su indicates a higher probability of RS≤25, wherein su is calculated by using the formula:

su=BASELINE+WF(PGR)·REL(PGR)−WF(MKI67)·REL(MKI67),

wherein WF(PGR) is a weighting factor for REL(PGR), and WF(MKI67) is a weighting factor for REL(MKI67).

In one embodiment, a higher score su indicates a higher probability of RS≤25, wherein su is calculated by using the formula:

su=25.490+0.847·REL(PGR)−1.353·REL(MKI67).

In one embodiment, a lower score su indicates a higher probability of RS≤25, wherein su is calculated by using the formula:

su=−BASELINE−WF(PGR)·REL(PGR)+WF(MKI67)·REL(MKI67),

wherein WF(PGR) is a weighting factor for REL(PGR), and WF(MKI67) is a weighting factor for REL(MKI67).

In one embodiment, a lower score su indicates a higher probability of RS≤25, wherein su is calculated by using the formula:

su=−25.490−0.847·REL(PGR)+1.353·REL(MKI67).

In one embodiment, the method further comprises:
calculating a predicted probability of RS≤25 q, wherein
a) if a higher score su indicates a higher probability of RS=25, q is calculated by using the formula

$$q = \frac{exp ( s u )}{( 1 + exp ( s u ) )} ;$$

and
b) if a lower score su indicates a higher probability of RS≤25, q is calculated by using the formula

$$q = 1 - \frac{exp ( s u )}{( 1 + exp ( s u ) )} ,$$

wherein, preferably, a q which is equal to or greater than a pre-defined threshold indicates a high probability of RS≤25, and a q which is lower than a pre-defined threshold indicates a low probability of RS≤25.

In one embodiment, the method further comprises:
calculating a clinical score s based on su, wherein s has a scale from 0 to 100 or from −10 to 10.

In one embodiment,
a) if a higher score su indicates a higher probability of RS≤25, a score s or a score su which is equal to or greater than a pre-defined threshold indicates a high probability of RS≤25, and a score s or a score su which is lower than the pre-defined threshold indicates a low probability of RS≤25; and
b) if a lower score su indicates a higher probability of RS≤25, a score s or a score su which is lower than a pre-defined threshold indicates a high probability of RS≤25, and a score s or a score su which is equal to or greater than the pre-defined threshold indicates a low probability of RS≤25.

In another aspect, the present invention relates to a method of predicting the probability of an Oncotype DX® low risk recurrence score (RS) result (RS≤25) for an ERBB2-negative breast cancer patient, said method comprising:
calculating a score unscaled (su) based on the relative expression levels of mRNA of ERBB2, ESR1, PGR and MKI67 in a breast tumor sample of the breast cancer patient as determined by reverse transcription quantitative PCR (RT-qPCR), wherein
a) a higher score su indicates a higher probability of RS≤25, wherein a higher relative expression level of mRNA of ERBB2 is associated with a lower su, a higher relative expression level of mRNA of ESR1 is associated with a higher su, a higher relative expression level of mRNA of PGR is associated with a higher su, and a higher relative expression level of mRNA of MKI67 is associated with a lower su; or
b) a lower score su indicates a higher probability of RS≤25, wherein a higher relative expression level of mRNA of ERBB2 is associated with a higher su, a higher relative expression level of mRNA of ESR1 is associated with a lower su, a higher relative expression level of mRNA of PGR is associated with a lower su, and a higher relative expression level of mRNA of MKI67 is associated with a higher su.

In one embodiment, the method comprises, prior to calculating su:
determining the relative expression levels of mRNA of ERBB2, ESR1, PGR and MKI67 in the breast tumor sample by RT-qPCR.

In another aspect, the present invention relates to a method for selecting a breast cancer treatment for an ERBB2-negative breast cancer patient, said method comprising:
predicting the probability of an Oncotype DX® low risk recurrence score (RS) result (RS≤25) for the breast cancer patient by using a method as defined above; and
selecting endocrine therapy as the breast cancer treatment for the breast cancer patient if a high probability of RS≤25 is predicted.

In one embodiment, a high probability of RS≤25 is predicted if su, q or s is higher than a pre-defined threshold.

In another aspect, the present invention relates to a method of treatment of ERBB2-negative breast cancer in a breast cancer patient comprising:
selecting a breast cancer treatment for the breast cancer patient by using a method as defined above; and
administering the selected breast cancer treatment to the breast cancer patient.

In another aspect, the present invention relates to an endocrine therapeutic compound for use in a method of treatment of ERBB2-negative breast cancer as defined above.

In another aspect, the present invention relates to the use of a kit in a method as defined above, wherein the kit comprises:
at least one pair of ERBB2-specific primers;
at least one pair of ESR1-specific primers;
at least one pair of PGR-specific primers; and/or
at least one pair of MKI67-specific primers.

In one embodiment, the kit further comprises at least one ERBB2-specific probe, at least one ESR1-specific probe, at least one PGR-specific probe and/or at least one MKI67-specific probe.

In one embodiment, the kit further comprises at least one pair of reference gene-specific primers and, optionally, at least one reference gene-specific probe.

In one embodiment, the reference gene is selected from the group consisting of B2M, CALM2, TBP, PUM1, MRLP19, GUSB, RPL37A and CYFIP1.

In another aspect, the present invention relates to a method of predicting the probability of an Oncotype DX® low risk recurrence score (RS) result (RS≤25) for a breast cancer patient as defined above, or a method for selecting a breast cancer treatment for a breast cancer patient as defined above, which is computer-implemented or partially computer-implemented.

In another aspect, the present invention relates to a data processing apparatus/device/system comprising means for carrying out the computer-implemented or partially computer-implemented method as defined above.

In another aspect, the present invention relates to a computer program comprising instructions which, when the program is executed by a computer, cause the computer to carry out the computer-implemented or partially computer-implemented method as defined above.

In another aspect, the present invention relates to a transitory or non-transitory, computer-readable data carrier having stored thereon the computer program as defined above.

### BRIEF DESCRIPTION OF THE FIGURES

FIG. 1 shows an ROC curve for unscaled score 1 in the training cohort.

FIG. 2 shows an ROC curve for unscaled score 1 in the validation cohort.

FIG. 3 shows the distribution of unscaled score values across different RS classes according to commercial and TailorX trial cut-offs (panel A: training cohort, N=202; panel B: validation cohort, N=104). Dotted line: cut-off defined on training set at 95% specificity (3.170). Solid line: cut-off defined on training set at 97.5% specificity (3.892).

FIG. 4 shows an ROC curve for rescaled score 1 (LRP score) in validation cohort 2.

FIG. 5 shows an ROC curve for rescaled score 1 (LRP score) against a simulated RS in ESR1-positive/ERBB2-negative samples.

FIG. 6 shows the distribution of the rescaled score 1 (LRP score) across different RS classes according to commercial and TailorX trial cut-offs (panel A: validation cohort 2, N=54; panel B: simulated RS cohort N=117 (ESR1-positive/ERBB2-negative)). Dotted line: cut-off defined on training set at 95% specificity (−0.722 on the rescaled score). Solid line: cut-off defined on training set at 100% specificity (0 on the rescaled score).

Other objects, advantages and features of the present invention will become apparent from the following detailed description, in particular when considered in conjunction with the accompanying figures.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

In the following, certain elements of the present invention will be described. These elements may be listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms (IUPAC Recommendations)”, H. G. W. Leuenberger, B. Nagel, and H. Kolb′, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 3^{rd} Edition, J. Sambrook et al. eds., Cold Spring Harbor Laboratory Press, Cold Spring Harbor 2000).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In one aspect, the present invention relates to a method of predicting the probability of an Oncotype DX® low risk recurrence score (RS) result (RS≤25) for an ERBB2-negative breast cancer patient, said method comprising:
calculating a score unscaled (su) based on the expression levels, preferably relative expression levels, of mRNA of ESR1, PGR and MKI67 in a breast tumor sample of the breast cancer patient as determined by reverse transcription quantitative PCR (RT-qPCR), wherein
a) a higher score su indicates a higher probability of RS≤25, wherein a higher expression level of mRNA of ESR1 is associated with a higher su, a higher expression level of mRNA of PGR is associated with a higher su, and a higher expression level of mRNA of MKI67 is associated with a lower su; or
b) a lower score su indicates a higher probability of RS≤25, wherein a higher expression level of mRNA of ESR1 is associated with a lower su, a higher expression level of mRNA of PGR is associated with a lower su, and a higher expression level of mRNA of MKI67 is associated with a higher su.

The term “breast cancer” relates to a type of cancer originating from breast tissue, most commonly from the inner lining of milk ducts or the lobules that supply the ducts with milk. Cancers originating from ducts are known as ductal carcinomas, while those originating from lobules are known as lobular carcinomas. Occasionally, breast cancer presents as metastatic disease. Common sites of metastasis include bone, liver, lung and brain. Breast cancer occurs in humans and other mammals. While the overwhelming majority of human cases occur in women, male breast cancer can also occur. In one embodiment of the present invention, the breast cancer is primary breast cancer (also referred to as early or early stage breast cancer). Primary breast cancer is breast cancer that hasn't spread beyond the breast or the lymph nodes under the arm. In one embodiment of the present invention, the breast cancer is an early stage, ERBB2-negative and ESR1-positive breast cancer which is either node-negative or node-positive.

The term “ERBB2-negative breast cancer” (also referred to as “HER2-negative breast cancer”) refers to a breast cancer with no or low expression levels of ERBB2, as determined by methods known in the art, e.g., by IHC and/or RT-qPCR.

The term “ESR1-positive breast cancer” refers to breast cancer with expression of ESR1, as determined by methods known in the art, e.g., by IHC and/or RT-qPCR. Such breast cancers may also be referred to as “hormone-receptor positive breast cancer”.

The term “tumor”, as used herein, refers to all neoplastic cell growth and proliferation whether malignant or benign, and all pre-cancerous and cancerous cells and tissues. The terms “tumor” and “cancer” may be used interchangeably herein. In one embodiment of the present invention, the tumor is a solid tumor.

Several molecular subtypes of breast cancer/tumors are known to the skilled person. The term “molecular subtype of a tumor” (or “molecular subtype of a cancer”), as used herein, refers to subtypes of a tumor/cancer that are characterized by distinct molecular profiles, e.g., gene expression profiles.

In one embodiment, the molecular subtype is selected from the group comprising, preferably consisting of, ERBB2/HER2-positive, triple-negative (also referred to as “basal-like”), luminal A(-like) and luminal B(-like). The term “basal-like” refers to the fact that such tumors have some similarity in gene expression to that of basal epithelial cells. The term “luminal” derives from the similarity in gene expression between the tumors and the luminal epithelium. In one embodiment, the molecular subtype is selected from the group comprising, preferably consisting of, the molecular subtypes according to the 13^{th} St Gallen guidelines (Goldhirsch A. et al., 2013), which are shown in below Table 1.

In one embodiment, the molecular subtype is determined by immunohistochemistry (IHC) on the protein level and/or by RT-qPCR on the mRNA level, preferably exclusively on the mRNA level, e.g., as described in WO 2015/024942 A1, which is incorporated herein by reference. In one embodiment, the molecular subtype, e.g., the molecular subtype according to the 13^{th} St Gallen guidelines, is determined by means of the MammaTyper® kit (BioNTech Diagnostics GmbH, Mainz, Germany; see also Laible M. et al., 2016), e.g., essentially as described in Example 2.

The term “patient”, as used herein, refers to a human or another mammal. Preferably, the patient is a human. Preferably, the patient is a female patient. In one embodiment, the patient is a postmenopausal female patient, which, preferably, is older than 50 years.

The Oncotype DX® recurrence score (RS) test is a well-known test that is routinely used for individualized risk assessment for breast cancer. It is included in clinical guidelines from organizations such as the American Society of Clinical Oncology (ASCO®), the National Comprehensive Cancer Network (NCCN®), the St. Gallen Consensus panel, the National Institute for Health Care Excellence (NICE), the European Society for Medical Oncology (ESMO) and the German Association of Gynecological Oncology (AGO). The test uses RT-PCR to measure the expression of 21 genes: 16 cancer-related genes and five reference genes. An RS≤25 test result indicates a low risk of recurrence and has been shown to indicate a lack of benefit from chemotherapy (Sparano et al., 2018).

The term “recurrence” with respect to cancer includes re-occurrence of tumor cells at the same site and organ of the origin disease, metastasis that can appear even many years after the initial diagnosis and therapy of cancer, or to local events such as infiltration of tumor cells into regional lymph nodes. “Distant recurrence” refers to a scenario, where the cancer cells have spread (metastasized) to a distant part (i.e., another organ) of the body beyond the regional lymph nodes. Recurrence-free survival is generally defined as the time from randomization to the first of recurrence, relapse, second cancer, or death.

The term “metastasis” is meant to refer to the spread of cancer cells from their original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential.

The term “mRNA” relates to “messenger RNA” and relates to a “transcript” which encodes a peptide or protein. mRNA typically comprises a 5′ non-translated region (5′-UTR), a protein or peptide coding region and a 3′ non-translated region (3′-UTR). mRNA has a limited halftime in cells and in vitro.

According to the present invention, the expression level of mRNA is determined by reverse transcription quantitative PCR (RT-qPCR). As RNA cannot be directly amplified in PCR, it must be reverse transcribed into cDNA using the enzyme reverse transcriptase. For this purpose, a one-step RT-qPCR can be utilized, which combines the reactions of reverse transcription with DNA amplification by PCR in the same reaction. In one-step RT-qPCR, the RNA template is mixed in a reaction mix containing reverse transcriptase, DNA polymerase, primers and probes, dNTPs, salts and detergents. In a first step, the target RNA is reverse transcribed by the enzyme reverse transcriptase using the target-specific reverse primers. Afterwards, the cDNA is amplified in a PCR reaction using the primers/probes and DNA polymerase.

In one embodiment, the quantitative PCR is fluorescence-based quantitative real-time PCR, in particular fluorescence-based quantitative real-time PCR. The fluorescence-based quantitative real-time PCR comprises the use of a fluorescently labeled probe. Preferably, the fluorescently labeled probe consists of an oligonucleotide labeled with both a fluorescent reporter dye and a quencher dye (=dual-label probe). Suitable fluorescent reporter and quencher dyes/moieties are known to a person skilled in the art and include, but are not limited to the reporter dyes/moieties 6-FAM™, JOE™, Cy5®, Cy3® and the quencher dyes/moieties dabcyl, TAMRA™, BHQ™-1, -2 or -3. Amplification of the probe-specific product causes cleavage of the probe (=amplification-mediated probe displacement), thereby generating an increase in reporter fluorescence. The increase of fluorescence in the reaction is directly proportional to the increase of target amplificates. By using a CFX96™ qPCR instrument (Bio-Rad) or a LightCycler® 480 II system (Roche Diagnostics) or a Versant kPCR system (Siemens) or a Mx3005P system (Agilent Technologies) or equivalent real-time instruments for detection of fluorescence originating from the probe, one can measure the increase in fluorescence in real-time. In one embodiment, the RT-qPCR is performed with a CFX96™ qPCR instrument (Bio-Rad). In another embodiment, RT-qPCR is performed with a qPCR system other than a CFX96™ qPCR system, and the results obtained with said system are mathematically transformed to correspond to the results obtained with the CFX96™ qPCR system. Analysis output is a Cq value (Cq=quantification cycle) for each target gene/sequence. The Cq value (also referred to as cycle threshold (CT) value) is determined by the number of PCR amplification cycles, after which the fluorescence signal of the probe exceeds a certain background signal, wherein the Cq value is a measure for the amount of target molecules in the sample before the PCR amplification. Preferably, Cq values are further analyzed with appropriate software (e.g., Microsoft Excel™) or statistical software packages (e.g., SAS JMP® 9.0.0, GraphPad Prism4, Genedata Expressionist™). The Cq value can either be converted to an absolute target molecule amount (e.g., ng/μl or molecules/μl) based on the Cq results of a standard curve with known target concentrations. Alternatively, the target amount can be reported as x-fold decreased or increased amount based on a reference (=ΔCq). Low ΔCq values (small difference) indicate higher amounts of target relative to the reference compared to high ΔCq (big difference). It is suitable to re-calculate the ΔCq by subtracting it from a fixed value (such as the number of PCR cycles, e.g., 40). The result is a value with direct correlation to target amount (high value=high amount) and expressed as 40-ΔCq values, wherein one integer refers to a doubling of the target amount (e.g., a value of 34 indicates an amount which is twice as much as that with a value of 33). Depending on the desired reproducibility and precision of the system, it is possible to panel multiple reference assays or to re-calculate/normalize the ΔCq of the sample with the ΔCq of a calibrator, resulting in a □□Cq value (1 point calibration; Pfaffl, 2001, Nucleic Acid Res. 29(9):e45). Preferably, Cq values are not transformed by any other mathematical operation which could skew the scale of the Cq values. By using different fluorophores for specific probes it is also possible to multiplex different target assays in the same reaction. During PCR, each target in the multiplex is amplified in parallel, but separately detected utilizing the different fluorescent emission.

In one embodiment, the term “expression level of mRNA”, as used herein, refers to the absolute expression level of mRNA, preferably given as Cq value. In one embodiment, the Cq value is used directly in calculations (e.g., subtraction from other Cq values) without prior normalization with one or more reference genes.

In another embodiment, the term “expression level of mRNA”, as used herein, refers to the relative expression level of mRNA.

In one embodiment, the amplification efficiency of the qPCR is from 90% to 110%. Preferably, if the amplification efficiency of the qPCR is below 90% or above 110%, the respective Cq values are corrected in order to be in accordance with a 100% amplification efficiency.

Preferably, primers for use in accordance with the present invention have a length of 15 to 30 nucleotides, in particular deoxyribonucleotides. In one embodiment, the primers are designed so as to (1) be specific for the target mRNA-sequence (e.g., ERBB2, ESR1, PGR or MKI67), (2) provide an amplicon size of less than 150 bp (preferably less than 100 bp), (3) detect all known protein-encoding splicing variants, (4) not include known polymorphisms (e.g., single nucleotide polymorphisms, SNPs), (5) be mRNA-specific (consideration of exons/introns; preferably no amplification of DNA), (6) have no tendency to dimerize and/or (7) have a melting temperature Tₘ in the range of from 58° C. to 62° C. (preferably, Tₘ is approximately 60° C.).

As used herein, the term “nucleotide” includes native (naturally occurring) nucleotides, which include a nitrogenous base selected from the group consisting of adenine (A), thymidine (T), cytosine (C), guanine (G) and uracil (U), a sugar selected from the group of ribose, arabinose, xylose, and pyranose, and deoxyribose (the combination of the base and sugar generally referred to as a “nucleoside”), and one to three phosphate groups, and which can form phosphodiester internucleosidyl linkages. Further, as used herein, “nucleotide” refers to nucleotide analogues. As used herein, “nucleotide analogue” shall mean an analogue of A, G, C, T or U (that is, an analogue of a nucleotide comprising the base A, G, C, T or U) which is recognized by DNA or RNA polymerase (whichever is applicable) and incorporated into a strand of DNA or RNA (whichever is appropriate). Examples of such nucleotide analogues include, without limitation, 5-propynyl pyrimidines (i.e., 5-propynyl-dTTP and 5-propynyl-dCTP), 7-deaza purines (i.e., 7-deaza-dATP and 7-deaza-dGTP), aminoallyl-dNTPs, biotin-AA-dNTPs, 2-amino-dATP, 5-methyl-dCTP, 5-iodo-dUTP, 5-bromo-dUTP, 5-fluoro-dUTP, N4-methyl-dCTP, 2-thio-dTTP, 4-thio-dTTP and alpha-thio-dNTPs. Also included are labelled analogues, e.g. fluorescent analogues such as DEAC-propylenediamine (PDA)-ATP, analogues based on morpholino nucleoside analogues as well as locked nucleic acid (LNA) analogues.

The wording “specific for the target mRNA-sequence”, as used in connection with primers for use in accordance with the present invention, is meant to refer to the ability of the primer to hybridize (i.e. anneal) to the cDNA of the target mRNA-sequence under appropriate conditions of temperature and solution ionic strength, in particular PCR conditions. The conditions of temperature and solution ionic strength determine the stringency of hybridization. Hybridization requires that the two nucleic acids (i.e. primer and cDNA) contain complementary sequences, although depending on the stringency of the hybridization, mismatches between bases are possible. In one embodiment, “appropriate conditions of temperature and solution ionic strength” refer to a temperature in the range of from 58° C. to 62° C. (preferably a temperature of approximately 60° C.) and a solution ionic strength commonly used in PCR reaction mixtures. In one embodiment, the sequence of the primer is 80%, preferably 85%, more preferably 90%, even more preferably 95%, 96%, 97%, 98%, 99% or 100% complementary to the corresponding sequence of the cDNA of the target mRNA-sequence, as determined by sequence comparison algorithms known in the art.

In one embodiment, the primer hybridizes to the cDNA of the target mRNA-sequence under stringent or moderately stringent hybridization conditions. “Stringent hybridization conditions”, as defined herein, involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). “Moderately stringent hybridization conditions”, as defined herein, involve including washing in 3×SSC at 42° C., or the art-recognized equivalent thereof. The parameters of salt concentration and temperature can be varied to achieve the optimal level of identity between the primer and the target nucleic acid. Guidance regarding such conditions is available in the art, for example, by J. Sambrook et al. eds., 2000, Molecular Cloning: A Laboratory Manual, 3^{rd} Edition, Cold Spring Harbor Laboratory Press, Cold Spring Harbor; and Ausubel et al. eds., 1995, Current Protocols in Molecular Biology, John Wiley and Sons, N.Y.

Preferably, probes for use in accordance with the present invention have a length of 20 to 35 nucleotides, in particular deoxyribonucleotides. In one embodiment, the probes are designed so as to (1) be specific for the target mRNA-sequence (e.g., ERBB2, ESR1, PGR or MKI67), (2) not include known polymorphisms (e.g., single nucleotide polymorphisms, SNPs) and/or (3) have a melting temperature Tₘ, which is approximately 5° C. to 8° C. higher than the melting temperature Tₘ of the corresponding primer(s).

The wording “specific for the target mRNA-sequence”, as used in connection with probes for use in accordance with the present invention, is meant to refer to the ability of the probe to hybridize (i.e. anneal) to the (amplified) cDNA of the target mRNA-sequence under appropriate conditions of temperature and solution ionic strength, in particular PCR conditions. The conditions of temperature and solution ionic strength determine the stringency of hybridization. Hybridization requires that the two nucleic acids (i.e. probe and cDNA) contain complementary sequences, although depending on the stringency of the hybridization, mismatches between bases are possible. In one embodiment, “appropriate conditions of temperature and solution ionic strength” refer to a temperature in the range of from 63° C. to 70° C. and a solution ionic strength commonly used in PCR reaction mixtures. In one embodiment, the sequence of the probe is 80%, preferably 85%, more preferably 90%, even more preferably 95%, 96%, 97%, 98%, 99% or 100% complementary to the corresponding sequence of the (amplified) cDNA of the target mRNA-sequence, as determined by sequence comparison algorithms known in the art.

In one embodiment, the probe hybridizes to the (amplified) cDNA of the target mRNA-sequence under stringent or moderately stringent hybridization conditions as defined above.

The probes as defined above are preferably labeled, e.g., with a label selected from a fluorescent label, a fluorescence quenching label, a luminescent label, a radioactive label, an enzymatic label and combinations thereof. Preferably, the probes as defined above are dual-label probes comprising a fluorescence reporter moiety and a fluorescence quencher moiety.

In one embodiment, the expression level is normalized against the (mean) expression level of one or more reference genes in the sample of the tumor. The term “reference gene”, as used herein, is meant to refer to a gene which has a relatively invariable level of expression on the RNA transcript/mRNA level in the system which is being examined, i.e. cancer. Such gene may be referred to as housekeeping gene. In one embodiment, the one or more reference genes are selected from the group comprising B2M, CALM2, TBP, PUM1, MRLP19, GUSB, RPL37A and CYFIP1. Other suitable reference genes are known to a person skilled in the art.

B2M refers to the gene of beta-2 microglobulin (UniProt: P61769), CALM2 refers to the gene of calmodulin-2 (UniProt: P0DP24), TBP refers to the gene of TATA-box-binding protein (UniProt: P20226), PUM1 refers to the gene of pumilio homolog 1 (UniProt: Q14671), MRLP19 refers to the gene of 39S ribosomal protein L19, mitochondrial (UniProt: P49406), GUSB refers to the gene of beta-glucuronidase (UniProt: P08236), RPL37A refers to the gene of Ribosomal Protein L37a (UniProt: P61513), and CYFIP1 refers to the gene of cytoplasmic FMR1-interacting protein 1 (UniProt: Q7L576).

In one embodiment, the primers for use in accordance with the present invention are selected from primers as described in WO 2015/024942 A1 and/or WO 2016/131875 A1, which are incorporated herein by reference. In one embodiment, the RT-qPCR is performed by means of the MammaTyper® kit (BioNTech Diagnostics GmbH, Mainz, Germany; see also Laible M. et al., 2016), e.g., essentially as described in Example 2.

The term “relative expression level (REL)”, as used herein, refers to the level of expression of a given marker gene (e.g., ERBB2, ESR1, PGR or MKI67) relative to the level of expression of one or more reference genes, e.g., one or more reference genes as defined herein. According to the present invention, the level of expression is determined on the mRNA level (transcriptional level) by RT-qPCR.

In one embodiment, the relative expression level (REL) is given as □Cq value which is calculated by subtracting the Cq value or mean/median Cq value of one or more reference genes from the Cq value or mean/median Cq value of the marker gene. In one embodiment, the □Cq value is further normalized by subtracting from said □Cq value the ΔCq value of a calibrator (e.g., a positive control, such as in vitro transcribed RNA of the marker gene), resulting in a Δ□Cq value.

In one embodiment, the relative expression level (REL) for a given marker gene, i.e., REL(ERBB2), REL (ESR1), REL(PGR) or REL(MKI67), is given as a value selected from the group consisting of □Cq value, □□Cq value, X-□Cq value and X-□Cq value, wherein, preferably, X is an integer, wherein, preferably, the integer is the number of PCR cycles of the RT-qPCR, e.g., 40. In one embodiment, REL is given as X-□□Cq value, e.g., 40-□□Cq value.

In one embodiment, the ΔCq value is calculated as follows: Cq of the respective marker (e.g., ERBB2, ESR1, PGR and/or MKI67) of a patient sample−Cq of a reference gene (e.g., B2M and/or CALM2) of a patient sample (=calculation method 1). In one embodiment, the Cq is the median/mean Cq. If more than one reference gene is used, the ΔCq value is calculated as follows: Cq of the respective marker of a patient sample−mean/median Cq of selected reference genes of a patient sample) (=calculation method 2).

In one embodiment, the ΔΔCq is calculated as follows: ΔΔCq=(Cq marker of a patient sample−Cq marker of a reference sample)−(Cq reference gene of patient sample−Cq reference gene of a reference sample) (=calculation method 3).

In another embodiment, the ΔΔCq value is calculated as follows: (Cq marker of a patient sample−Cq reference gene of the patient sample)−(Cq marker of a control sample−Cq reference gene of the control sample)] (=calculation method 4). In one embodiment, the Cq is the median/mean Cq. The Cq of the reference gene can be the Cq of a single reference gene or the mean Cq of two or more reference genes (referred to as mean/median CombRef). Preferably, the same control sample (also referred to as calibrator) is used in all analyses and leads to the same RT-qPCR or qPCR results. In one embodiment, the calibrator is a positive control (PC). In one embodiment, the control sample is a cell line RNA, an in vitro transcribed RNA or an equimolar mixture of DNA oligonucleotides, representing the marker mRNA or cDNA or the marker amplicon or a part of the marker amplicon with a constant ratio. In one embodiment, CALM2 and/or B2M are used as reference genes and a positive control, e.g., in vitro transcribed RNA, is used as control sample (calibrator).

The gene ERBB2 (also referred to as HER2; location: 17q12, annotation: chromosome: 17; NC_000017.10; UniProt: P04626) encodes a member of the epidermal growth factor (EGF) receptor family of receptor tyrosine kinases. Amplification and/or overexpression of this gene have been reported in numerous cancers, including breast and ovarian tumors. In the NCBI database, two mRNA variants for ERBB2 are listed which code for two protein versions. Protein and mRNA sequences can be found under the accession numbers NM 001005862.1 (receptor tyrosine-protein kinase erbB-2 isoform b) and NM_004448.2 (receptor tyrosine-protein kinase erbB-2 isoform a precursor).

The gene ESR1 (location: 6q25, annotation: chromosome 6, NC_000006.11; UniProt: P03372) encodes an estrogen receptor (ER), a ligand-activated transcription factor composed of several domains important for hormone binding, DNA binding, and activation of transcription. Estrogen receptors are known to be involved in pathological processes including breast cancer, endometrial cancer, and osteoporosis. Four ESR1 mRNA variants are known, wherein the transcript variants differ in the 5′ UTR and/or use different promoters, but each variant codes for the same protein.

The gene PGR (also referred to as PR; location: 11q22-q23, annotation: chromosome: 11; NC_000011.9; UniProt: P06401) encodes the progesterone receptor. Steroid hormones such as progesterone and their receptors are involved in the regulation of eukaryotic gene expression and affect cellular proliferation and differentiation in target tissues. This gene uses two distinct promoters and translation start sites in the first exon to produce two mRNA isoforms, A and B. The two isoforms are identical except for the additional 165 amino acids found in the N-terminus of isoform B.

The gene MKI67 (also referred to as Ki67; location: 10q26.2, annotation: chromosome: 10; NC_000010.10; UniProt: P46013) encodes a nuclear protein that is associated with and may be necessary for cellular proliferation. Two mRNA variants have been described. A related pseudogene exists on chromosome 10.

In one embodiment of the present invention, the term “breast tumor sample” refers to a breast tumor tissue sample isolated from the cancer patient (e.g., a biopsy or resection tissue of the breast tumor). In a preferred embodiment, the breast tumor tissue sample is a cryo-section of a breast tumor tissue sample or is a chemically fixed breast tumor tissue sample. In a more preferred embodiment, the breast tumor tissue sample is a formalin-fixed and paraffin-embedded (FFPE) breast tumor tissue sample. In one embodiment, the sample of the breast tumor is (total) RNA extracted from the breast tumor tissue sample. In a particularly preferred embodiment, the sample of the breast tumor is (total) RNA extracted from a FFPE breast tumor tissue sample. In another embodiment, the breast tumor sample is a sample of one or more circulating tumor cells (CTCs) or (total) RNA extracted from the one or more CTCs. Those skilled in the art are able to perform RNA extraction procedures. For example, total RNA from a 5 to 10 μm curl of FFPE tumor tissue can be extracted using the High Pure RNA Paraffin kit (Roche, Basel, Switzerland), the XTRAKT RNA Extraction kit XL (Stratifyer Molecular Pathology, Cologne, Germany) or the RNXtract® Extraction kit (BioNTech Diagnostics GmbH, Mainz, Germany). It is also possible to store the sample material to be used/tested in a freezer and to carry out the methods of the present invention at an appropriate point in time after thawing the respective sample material. In one embodiment, the breast tumor sample is a pre-treatment breast tumor sample, i.e., a breast tumor sample which is obtained from the breast cancer patient prior to initiation/administration of breast cancer treatment.

The term “treatment”, in particular in connection with the treatment of cancer, as used herein, relates to any treatment which improves the health status and/or prolongs (increases) the lifespan of a patient. Said treatment may eliminate cancer, reduce the size or the number of tumors in a patient, arrest or slow the development of cancer in a patient, inhibit or slow the development of new cancer in a patient, decrease the frequency or severity of symptoms in a patient, and/or decrease recurrences in a patient who currently has or who previously has had cancer.

The term “breast cancer treatment”, as used herein, may include surgery, medications (anti-hormonal/endocrine therapy and chemotherapy), radiation, immunotherapy/targeted therapy as well as combinations of any of the foregoing.

Endocrine therapy (also referred to as “anti-hormonal therapy” or “anti-hormone” therapy), as used herein, refers to treatment that blocks or removes hormones. Endocrine therapy targets cancers that require estrogen to continue growing by administration of drugs that either block/down-regulate estrogen and/or progesterone receptors, e.g., tamoxifen (Nolvadex®) or fulvestrant (Faslodex®), or alternatively block the production of estrogen with an aromatase inhibitor, e.g., anastrozole (Arimidex®) or letrozole (Femara®). Aromatase inhibitors, however, are only suitable for post-menopausal patients. This is because the active aromatase in postmenopausal women is different from the prevalent form in premenopausal women, and therefore these agents are ineffective in inhibiting the predominant aromatase of premenopausal women. The term “endocrine therapeutic compound” or “endocrine therapeutic agent”, as used herein, is meant to refer to a compound/agent/drug that blocks or removes hormones upon administration to a patient, in particular a compound/agent/drug that either blocks/down-regulates estrogen and/or progesterone receptors or blocks the production of estrogen and/or progesterone. Exemplary compounds/agents/drugs include, but are not limited to, tamoxifen (Nolvadex®), fulvestrant (Faslodex®) and aromatase inhibitors, such as anastrozole (Arimidex®) and letrozole (Femara®). In one embodiment, endocrine therapy comprises administration of an aromatase inhibitor.

Chemotherapy comprises the administration of chemotherapeutic agents. Chemotherapeutic agents or compounds according to the invention include cytostatic compounds and cytotoxic compounds. Traditional chemotherapeutic agents act by killing cells that divide rapidly, one of the main properties of most cancer cells. According to the invention, the term “chemotherapeutic agent” or “chemotherapeutic compound” includes taxanes, platinum compounds, nucleoside analogs, camptothecin analogs, anthracyclines and anthracycline analogs, etoposide, bleomycin, vinorelbine, cyclophosphamide, antimetabolites, anti-mitotics, and alkylating agents, including the agents disclosed above in connection with antibody conjugates, and combinations thereof. In one embodiment, the chemotherapy is platinum-based, i.e. comprises the administration of platinum-based compounds, e.g., cisplatin. According to the invention a reference to a chemotherapeutic agent is to include any prodrug such as ester, salt or derivative such as a conjugate of said agent. Examples are conjugates of said agent with a carrier substance, e.g., protein-bound paclitaxel such as albumin-bound paclitaxel. Preferably, salts of said agent are pharmaceutically acceptable. Chemotherapeutic agents are often given in combinations, usually for 3-6 months. One of the most common treatments is cyclophosphamide plus doxorubicin (adriamycin; belonging to the group of anthracyclines and anthracycline analogs), known as AC. Sometimes, a taxane drug, such as docetaxel, is added, and the regime is then known as CAT; taxane attacks the microtubules in cancer cells. Thus, in one embodiment, chemotherapy, e.g., neo-adjuvant chemotherapy, comprises administration of cyclophosphamide, an anthracycline and a taxane. Another common treatment, which produces equivalent results, is cyclophosphamide, methotrexate, which is an antimetabolite, and fluorouracil, which is a nucleoside analog (CMF). Another standard chemotherapeutic treatment comprises fluorouracil, epirubicin and cyclophosphamide (FEC), which may be supplemented with a taxane, such as docetaxel, or with vinorelbine.

In one embodiment, the term “anti-ERBB2 drug”, as used herein, refers to anti-ERBB2/HER2 antibodies, in particular monoclonal anti-ERBB2/HER2 antibodies. Monoclonal anti-ERBB2/HER2 antibodies include trastuzumab (Herceptin®) and pertuzumab (Perjeta®), which may be administered alone or in combination. A combination of trastuzumab and pertuzumab is also referred to as “dual blockade” of ERBB2/HER2. Trastuzumab is effective only in cancers where ERBB2/HER2 is overexpressed. Other monoclonal antibodies, such as ertumaxomab (Rexomun®), are presently undergoing clinical trials. The anti-ERBB2/HER2 antibodies can further be modified to comprise a therapeutic moiety/agent, such as a cytotoxic agent, a drug (e.g., an immunosuppressant), a chemotherapeutic agent or a radionuclide, or a radioisotope. Thus, if the tumor treatment regimen comprises (a combination of) anti-ERBB2/HER2 therapy and chemotherapy, an anti-ERBB2/HER2 antibody conjugated to a chemotherapeutic agent may be used. A cytotoxin or cytotoxic agent includes any agent that is detrimental to and, in particular, kills cells. Examples include mertansine or emtansine (DM1), taxol, cytochalasin B, gramicidin D, ethidium bromide, emetine, mitomycin, etoposide, tenoposide, vincristine, vinblastine, colchicin, doxorubicin, daunorubicin, dihydroxy anthracin, dione, mitoxantrone, mithramycin, actinomycin D, amanitin, 1-dehydrotestosterone, glucocorticoids, procaine, tetracaine, lidocaine, propranolol, and puromycin and analogs or homologs thereof. In one embodiment, the antibody conjugate is trastuzumab (T)-DM1, e.g., trastuzumab emtansine. Other suitable therapeutic agents for forming antibody conjugates include, but are not limited to, antimetabolites (e.g., methotrexate, 6-mercaptopurine, 6-thioguanine, cytarabine, fludarabin, 5-fluorouracil decarbazine), alkylating agents (e.g., mechlorethamine, thioepachlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In another preferred embodiment, the therapeutic agent is doxorubicin, cisplatin, bleomycin, sulfate, carmustine, chlorambucil, cyclophosphamide or ricin A. Further therapeutic moieties include therapeutic moieties acting on mRNA and/or protein synthesis. Several inhibitors of transcription are known. For instance, actinomycin D, which is both a transcriptional inhibitor and a DNA damage agent, intercalates within the DNA and thus inhibits the initiation stage of transcription. Flavopiridol targets the elongation stage of transcription. alpha-Arnanitin binds directly to RNA polymerase II, which leads to the inhibition of both initiation and elongation stages. Anti-ERBB2/HER2 antibodies also can be conjugated to a radioisotope, e.g., iodine-131, yttrium-90 or indium-111, to generate cytotoxic radiopharmaceuticals. In another embodiment, the term “anti-ERBB2 drug”, as used herein, refers to small compounds targeting ERBB2/HER2, such as lapatinib (Tykerb® or Tyverb®), afatinib or neratinib.

Adjuvant therapy is a treatment that is given in addition to (i.e., after) the primary, main or initial treatment. An example of adjuvant therapy is the additional treatment (e.g., by chemotherapy) given after surgery (post-surgically), wherein, preferably, all detectable disease has been removed, but where there remains a statistical risk of relapse due to occult disease. Neo-adjuvant therapy is treatment given before the main treatment, e.g., chemotherapy before surgery (pre-surgical chemotherapy). In one embodiment, the term “chemotherapy”, as used herein, refers to adjuvant chemotherapy. In another embodiment, the term “chemotherapy”, as used herein, refers to neo-adjuvant chemotherapy.

In one embodiment, the method comprises, prior to calculating su:
determining the expression levels, preferably relative expression levels, of mRNA of ESR1, PGR and MKI67 in the breast tumor sample by RT-qPCR.

In one embodiment, no expression level, preferably no relative expression level, of mRNA of a gene other than ESR1, PGR and MKI67, and, optionally, one or more reference genes is determined.

In one embodiment, the breast cancer is an ERBB2-negative and ESR1-positive breast cancer.

In one embodiment, in the calculation of su, the relative expression levels (RELs) of mRNA of ESR1, PGR and MKI67 are weighted as follows:

REL(ESR1):REL(PGR):REL(MKI67)=0.60(±0.09):1(±0.15):1.78(±0.27).

In one embodiment, a higher score su indicates a higher probability of RS≤25, wherein su is calculated by using the formula:

su=BASELINE+WF(ESR1)·REL(ESR1)+WF(PGR)·REL(PGR)−WF(MKI67)·REL(MKI67),

wherein WF(ESR1) is a weighting factor for REL(ESR1), WF(PGR) is a weighting factor for REL(PGR), and WF(MKI67) is a weighting factor for REL(MKI67).

In one embodiment, a higher score su indicates a higher probability of RS≤25, wherein su is calculated by using the formula:

su=12.313+0.539·REL(ESR1)+0.902·REL(PGR)−1.602·REL(MKI67).

In one embodiment, a lower score su indicates a higher probability of RS≤25, wherein su is calculated by using the formula:

su=−BASELINE−WF(ESR1)·REL(ESR1)−WF(PGR)·REL(PGR)+WF(MKI67)·REL(MKI67),

wherein WF(ESR1) is a weighting factor for REL(ESR1), WF(PGR) is a weighting factor for REL(PGR), and WF(MKI67) is a weighting factor for REL(MKI67).

In one embodiment, a lower score su indicates a higher probability of RS≤25, wherein su is calculated by using the formula:

su=−12.313−0.539·REL(ESR1)−0.902·REL(PGR)+1.602·REL(MKI67).

In one embodiment, the method further comprises:
calculating a predicted probability of RS≤25 q, wherein
a) if a higher score su indicates a higher probability of RS≤25, q is calculated by using the formula

$$q = \frac{exp ( s u )}{( 1 + exp ( s u ) )} ;$$

and
b) if a lower score su indicates a higher probability of RS≤25, q is calculated by using the formula

$$q = 1 - \frac{exp ( s u )}{( 1 + exp ( s u ) )} ,$$

wherein, preferably, a q which is equal to or greater than a pre-defined threshold indicates a high probability of RS≤25, and a q which is lower than a pre-defined threshold indicates a low probability of RS≤25.

In one embodiment, the method further comprises:
calculating a clinical score s based on su, wherein s has a scale from 0 to 100 or from −10 to 10.

In one embodiment, the clinical score s is calculated by subtracting a pre-defined threshold/cut-off from su, wherein, preferably, s has a scale from −10 to 10.

In one embodiment,
a) if a higher score su indicates a higher probability of RS≤25, a score s or a score su which is equal to or greater than a pre-defined threshold indicates a high probability of RS≤25, and a score s or a score su which is lower than the pre-defined threshold indicates a low probability of RS≤25; and
b) if a lower score su indicates a higher probability of RS≤25, a score s or a score su which is lower than a pre-defined threshold indicates a high probability of RS≤25, and a score s or a score su which is equal to or greater than the pre-defined threshold indicates a low probability of RS≤25.

Suitable baselines for use in the formulae described herein as well as pre-defined thresholds/cut-offs, e.g., thresholds/cut-offs for dichotomization of scores in “low probability of RS≤25” or “high probability of RS≤25”, can be readily determined by the skilled person based on his or her general knowledge and the technical guidance provided herein (see Examples). For example, concordance studies in a training-testing setting can be used for the definition and validation of suitable thresholds/cut-offs. In one embodiment, the thresholds/cut-offs are defined based on one or more previous clinical studies. Moreover, additional clinical studies may be conducted for the establishment and validation of the thresholds/cut-offs. The thresholds/cut-offs may be determined/defined by techniques known in the art. In one embodiment, the thresholds/cut-offs are determined/defined on the basis of the data for RS≤25 in training cohorts by partitioning tests, ROC analyses or other statistical methods (e.g., by using the SAS Software JMP® 9.0.0).

In another aspect, the present invention relates to a method of predicting the probability of an Oncotype DX® low risk recurrence score (RS) result (RS≤25) for an ERBB2-negative breast cancer patient, said method comprising:
calculating a score unscaled (su) based on the expression levels, preferably relative expression levels, of mRNA of PGR and MKI67 in a breast tumor sample of the breast cancer patient as determined by reverse transcription quantitative PCR (RT-qPCR), wherein
a) a higher score su indicates a higher probability of RS≤25, wherein a higher expression level of mRNA of PGR is associated with a higher su, and a higher expression level of mRNA of MKI67 is associated with a lower su; or
b) a lower score su indicates a higher probability of RS≤25, wherein a higher expression level of mRNA of PGR is associated with a lower su, and a higher expression level of mRNA of MKI67 is associated with a higher su.

In one embodiment, the method comprises, prior to calculating su:
determining the expression levels, preferably relative expression levels, of mRNA of PGR and
   MKI67 in the breast tumor sample by RT-qPCR.

In one embodiment, no expression level, preferably no relative expression level, of mRNA of a gene other than PGR and MKI67, and, optionally, one or more reference genes is determined.

In one embodiment, the breast cancer is an ERBB2-negative and ESR1-positive breast cancer.

In one embodiment, in the calculation of su, the relative expression levels (RELs) of mRNA of PGR and MKI67 are weighted as follows:

REL(PGR):REL(MKI67)=1(±0.15):1.60(±0.24).

In one embodiment, a higher score su indicates a higher probability of RS≤25, wherein su is calculated by using the formula:

su=BASELINE+WF(PGR)·REL(PGR)−WF(MKI67)·REL(MKI67),

wherein WF(PGR) is a weighting factor for REL(PGR), and WF(MKI67) is a weighting factor for REL(MKI67).

In one embodiment, a higher score su indicates a higher probability of RS≤25, wherein su is calculated by using the formula:

su=25.490+0.847·REL(PGR)−1.353·REL(MKI67).

In one embodiment, a lower score su indicates a higher probability of RS≤25, wherein su is calculated by using the formula:

su=−BASELINE−WF(PGR)·REL(PGR)+WF(MKI67)·REL(MKI67),

wherein WF(PGR) is a weighting factor for REL(PGR), and WF(MKI67) is a weighting factor for REL(MKI67).

In one embodiment, a lower score su indicates a higher probability of RS≤25, wherein su is calculated by using the formula:

su=−25.490−0.847·REL(PGR)+1.353·REL(MKI67).

In one embodiment, the method further comprises:
calculating a predicted probability of RS≤25 q, wherein
a) if a higher score su indicates a higher probability of RS≤25, q is calculated by using the formula

$$q = \frac{exp ( s u )}{( 1 + exp ( s u ) )} ;$$

and
b) if a lower score su indicates a higher probability of RS≤25, q is calculated by using the formula

$$q = 1 - \frac{exp ( s u )}{( 1 + exp ( s u ) )} ,$$

wherein, preferably, a q which is equal to or greater than a pre-defined threshold indicates a high probability of RS≤25, and a q which is lower than a pre-defined threshold indicates a low probability of RS≤25.

In one embodiment, the method further comprises:
calculating a clinical score s based on su, wherein s has a scale from 0 to 100 or from −10 to 10.

In one embodiment, the clinical score s is calculated by subtracting a pre-defined threshold/cut-off from su, wherein, preferably, s has a scale from −10 to 10.

In one embodiment,
a) if a higher score su indicates a higher probability of RS≤25, a score s or a score su which is equal to or greater than a pre-defined threshold indicates a high probability of RS≤25, and a score s or a score su which is lower than the pre-defined threshold indicates a low probability of RS≤25; and
b) if a lower score su indicates a higher probability of RS≤25, a score s or a score su which is lower than a pre-defined threshold indicates a high probability of RS≤25, and a score s or a score su which is equal to or greater than the pre-defined threshold indicates a low probability of RS≤25.

In another aspect, the present invention relates to a method of predicting the probability of an Oncotype DX® low risk recurrence score (RS) result (RS≤25) for an ERBB2-negative breast cancer patient, said method comprising:
calculating a score unscaled (su) based on the expression levels, preferably relative expression levels, of mRNA of ERBB2, ESR1, PGR and MKI67 in a breast tumor sample of the breast cancer patient as determined by reverse transcription quantitative PCR (RT-qPCR), wherein
a) a higher score su indicates a higher probability of RS≤25, wherein a higher expression level of mRNA of ERBB2 is associated with a lower su, a higher expression level of mRNA of ESR1 is associated with a higher su, a higher expression level of mRNA of PGR is associated with a higher su, and a higher expression level of mRNA of MKI67 is associated with a lower su; or
b) a lower score su indicates a higher probability of RS≤25, wherein a higher expression level of mRNA of ERBB2 is associated with a higher su, a higher expression level of mRNA of ESR1 is associated with a lower su, a higher expression level of mRNA of PGR is associated with a lower su, and a higher expression level of mRNA of MKI67 is associated with a higher su.

In one embodiment, the method comprises, prior to calculating su:
determining the expression levels, preferably relative expression levels, of mRNA of ERBB2, ESR1, PGR and MKI67 in the breast tumor sample by RT-qPCR.

In one embodiment, no expression level, preferably no relative expression level, of mRNA of a gene other than ERBB2, ESR1, PGR and MKI67, and, optionally, one or more reference genes is determined.

In another aspect, the present invention relates to a method for selecting a breast cancer treatment for an ERBB2-negative breast cancer patient, said method comprising:
predicting the probability of an Oncotype DX® low risk recurrence score (RS) result (RS≤25) for the breast cancer patient by using a method as defined above; and
selecting endocrine therapy as the breast cancer treatment for the breast cancer patient if a high probability of RS≤25 is predicted.

In one embodiment, a high probability of RS≤25 is predicted if su, q or s is higher than a pre-defined threshold.

In one embodiment, if a high probability of RS≤25 is predicted, the breast cancer patient is excluded from chemotherapy.

In one embodiment, the methods of the invention as defined above do not comprise any other diagnostic steps, such as histological tumor grading or determining the (axillary) lymph nodal status. In one embodiment, the methods do not comprise any steps involving immunohistochemistry (IHC).

In one embodiment, the methods of the invention further comprise the consideration of one or more clinical factors, such as histological tumor grade, (axillary) lymph-nodal status, tumor size, age of the patient etc.

In another aspect, the present invention relates to a method of treatment of ERBB2-negative breast cancer in a breast cancer patient comprising:
selecting a breast cancer treatment for the breast cancer patient by using a method as defined above; and
administering the selected breast cancer treatment to the breast cancer patient.

In another aspect, the present invention relates to a endocrine therapeutic compound for use in a method of treatment of ERBB2-negative breast cancer as defined above.

In another aspect, the present invention relates to the use of a kit in a method as defined above, wherein the kit comprises:
at least one pair of ERBB2-specific primers;
at least one pair of ESR1-specific primers;
at least one pair of PGR-specific primers; and/or at least one pair of MKI67-specific primers.

In one embodiment, the kit comprises:
at least one pair of PGR-specific primers; and
at least one pair of MKI67-specific primers.

In one embodiment, the kit comprises:
at least one pair of ESR1-specific primers;
at least one pair of PGR-specific primers; and
at least one pair of MKI67-specific primers.

In one embodiment, the kit comprises:
at least one pair of ERBB2-specific primers;
at least one pair of ESR1-specific primers;
at least one pair of PGR-specific primers; and
at least one pair of MKI67-specific primers.

In one embodiment, the kit further comprises at least one ERBB2-specific probe, at least one ESR1-specific probe, at least one PGR-specific probe and/or at least one MKI67-specific probe. In one embodiment, the kit comprises at least one PGR-specific probe and at least one MKI67-specific probe. In one embodiment, the kit further comprises at least one ESR1-specific probe, at least one PGR-specific probe and at least one MKI67-specific probe. In one embodiment, the kit further comprises at least one ERBB2-specific probe, at least one ESR1-specific probe, at least one PGR-specific probe and at least one MKI67-specific probe.

In one embodiment, the kit further comprises at least one pair of reference gene-specific primers and, optionally, at least one reference gene-specific probe. In one embodiment, the reference gene is selected from the group consisting of B2M, CALM2, TBP, PUM1, MRLP19, GUSB, RPL37A and CYFIP1. In one embodiment, B2M and/or CALM2 are used as references genes.

Preferably, the primers and/or the probes are as defined further above. In one embodiment, the primers provide an amplicon size of less than 150 bp, preferably less than 100 bp. In one embodiment, detection of the probe is based on amplification-mediated probe displacement. In one embodiment, the probe is a dual-label probe comprising a fluorescence reporter moiety and a fluorescence quencher moiety.

In one embodiment, the kit does not comprise any primers and/or probes that are specific for additional non-reference genes. In other words, no primers and/or probes specific for a gene other than ERBB2, ESR1, PGR and MKI67, and, optionally, one or more reference genes is comprised in the kit. In one embodiment, no primers and/or probes specific for a gene other than ERBB2, ESR1, MKI67, and, optionally, one or more reference genes is comprised in the kit. In another embodiment, no primers and/or probes specific for a gene other than ESR1 and MKI67, and, optionally, one or more reference genes is comprised in the kit.

In one embodiment, the kit further comprises at least one control RNA sample. In one embodiment, the at least one control RNA sample is used as a positive control and/or a control sample (calibrator), wherein, preferably, the at least one control RNA sample comprises synthetic mRNA coding for one or more gene products (or parts thereof) of one or more genes selected from the group comprising ERBB2, ESR1, PGR, MKI67 and one or more reference genes. In one embodiment, the one or more reference genes are selected from the group consisting of B2M, CALM2, TBP, PUM1, MRLP19, GUSB, RPL37A and CYFIP1. In one embodiment, B2M and/or CALM2 are used as references genes.

In one embodiment, the kit further comprises a reverse transcriptase and a DNA polymerase. In one embodiment, the reverse transcriptase and the DNA polymerase are provided in the form of an enzyme-mix which allows a one-step RT-qPCR.

In one embodiment, the kit may further comprise a DNase and a DNase reaction buffer.

As used herein, the term “kit of parts (in short: kit)” refers to an article of manufacture comprising one or more containers and, optionally, a data carrier. Said one or more containers may be filled with one or more of the above mentioned means or reagents. Additional containers may be included in the kit that contain, e.g., diluents, buffers and further reagents such as dNTPs. Said data carrier may be a non-electronical data carrier, e.g., a graphical data carrier such as an information leaflet, an information sheet, a bar code or an access code, or an electronical/computer-readable data carrier such as a compact disk (CD), a digital versatile disk (DVD), a microchip or another semiconductor-based electronical data carrier. The access code may allow the access to a database, e.g., an internet database, a centralized, or a decentralized database. Said data carrier may comprise instructions for the use of the kit in the methods of the invention. The data carrier may comprise threshold values or reference levels of (relative) expression levels of mRNA or of the scores calculated according to the methods of the present invention. In case that the data carrier comprises an access code which allows the access to a database, said threshold values or reference levels are deposited in this database. In addition, the data carrier may comprise information or instructions on how to carry out the methods of the present invention.

In one embodiment, the kit is the MammaTyper® kit (BioNTech Diagnostics GmbH, Mainz, Germany; see also Laible M. et al., 2016).

In another aspect, the present invention relates to a method of predicting the probability of an Oncotype DX® low risk recurrence score (RS) result (RS≤25) for a breast cancer patient as defined above, or a method for selecting a breast cancer treatment for a breast cancer patient as defined above, which is computer-implemented or partially computer-implemented.

The term “partially computer-implemented method” refers to a method in which only particular steps, e.g., calculating steps, are computer-implemented, whereas other steps of the method are not.

In another aspect, the present invention relates to a data processing apparatus/device/system comprising means for carrying out the computer-implemented or partially computer-implemented method as defined above.

In another aspect, the present invention relates to a computer program comprising instructions which, when the program is executed by a computer, cause the computer to carry out the computer-implemented or partially computer-implemented method as defined above.

In another aspect, the present invention relates to a transitory or non-transitory, computer-readable data carrier having stored thereon the computer program as defined above.

The present inventors have surprisingly shown that the mRNA expression levels of a four-marker set (ERBB2, ESR1, PGR, MKI67) or a three-marker set (ESR1, PGR, MKI67) or a two-marker set (PGR, MKI67), as determined by RT-qPCR, can serve as reliable predictors of an Oncotype DX® low risk recurrence score (RS) result (RS≤25) for a breast cancer patient, in particular a patient having an ERBB2-negative and ESR1-positive primary breast cancer, thereby making the performance of an additional Oncotype DX® RS test obsolete.

The present invention is further illustrated by the following examples which are not be construed as limiting the scope of the invention.

### EXAMPLES

### Example 1: Isolating Total RNA from FFPE Samples Using the RNXtract® Protocol

Fixation of tumor tissue with formalin and subsequent embedding in paraffin is a standard method in clinical pathology and allows long-term archiving of samples. Because of chemical modifications of nucleic acids in FFPE samples, special protocols are necessary to extract amplifiable nucleic acids. Three steps are required for this: (1) removal of the paraffin, (2) lysis of the tissue and release of RNA (de-modification of nucleic acids if required), (3) purification of RNA by several washing steps.

The RNXtract® kit (BioNTech Diagnostics GmbH, Mainz, Germany) allows purification without organic solvents, which can be conducted in a single reaction vessel.

In the first step, the paraffin contained in the FFPE sections is liquefied in an optimized lysis buffer. Subsequent addition of proteinase K leads to lysis of the tissue and release of cellular nucleic acids (RNA and DNA). The RNA is bound to magnetic particles, which are functionalized with germanium and allow a very efficient binding of RNA, within a binding buffer optimized for efficient enrichment of RNA. The RNA bound to magnetic particles is then washed in several increasingly stringent washing steps to ensure efficient removal of proteins and PCR-inhibiting substances, and is subsequently eluted in elution buffer. The eluate can be used directly in suitable molecular biological analyses such as reverse transcription, RT-qPCR, microarrays or NGS-applications. Quantification by RT-qPCR methods or UV/VIS spectrophotometry is possible. For use of RNXtract® eluate in a MammaTyper® RTqPCR (see below) no digestion of potential residual RNA is required.

### Example 2: Measuring the Gene Expression Level of the Biomarkers Using the MammaTyper® Kit

The MammaTyper® kit (BioNTech Diagnostics GmbH, Mainz, Germany) allows the determination of the level of expression of selected biomarkers at the mRNA level by means of reverse transcription quantitative PCR (RT-qPCR).

To determine the expression level of a biomarker at transcript level by PCR, RNA has first to be transcribed into complementary DNA (cDNA) via the enzyme reverse transcriptase (so-called first strand synthesis). The marker-specific cDNA is then amplified by a DNA polymerase and amplification is detected in the PCR in real time using fluorescently labeled hydrolysis probes. The RT-qPCR takes place as a one-step reaction in the MammaTyper® assay, i.e., reverse transcription of the RNA and subsequent PCR of the DNA occur consecutively in the same reaction mixture. In addition to the enzymes (reverse transcriptase and DNA polymerase), the enzyme mix contains dNTPs as well as salts and PCR additives. For a MammaTyper® RT-qPCR, the enzyme mix is supplemented with water, assay mix and the RNA sample.

In each of the three assay mixes, two assays (assay=primer pair and probe specific for the respective target sequence) are combined (=duplexed). Simultaneous detection of the two targets in the duplexed assays is realized using hydrolysis probes with different fluorophore-labeling; in each assay mix, detection is carried out using FAM in one assay and JOE in the other assay. Hydrolysis probes are modified with the respective fluorescent dye at the 5′ end and a quencher at the 3′ end. The quencher suppresses the fluorescence of the dye as long as it is in close proximity to the dye. In the course of amplification the probe binds to the target sequence. Due to the exonuclease activity of DNA polymerase, the bound probe is degraded and the dye and quencher are separated. The resulting fluorescence measured at the end of each cycle is directly proportional to the amount of synthesized product. In real-time PCR assays using hydrolysis probes, the number of PCR cycles required to obtain a fluorescence signal bigger than the background signal is used as a measure of the number of existing target molecules at the beginning of the reaction. The PCR cycle at which a signal can be detected above the background signal is referred to as the quantification cycle (Cq). In the relative expression analysis, the difference of the Cq values of the target assay and the reference assay (=ΔCq) is determined to compensate for variations in the amount of RNA starting material. In addition, the ΔCq value is offset against a calibrator to correct for inter-run and inter-instrument variations (ΔΔCq) for different instruments of one manufacturer.

Marker-specific primers and probes are selected in a way that no amplification and/or detection occurs without target gene RNA or with undesirable sequences or analytes (e.g., genomic DNA), whereas the target gene of interest is detected sensitively. Suitable primers are described, for example, in WO 2015/024942 A1 and/or WO 2016/131875 A1.

Using the MammaTyper® kit at least one patient sample is analyzed per RT-qPCR run. Additionally, external controls are analyzed within each run, which determine the validity/invalidity of the run. For this purpose, a positive control RNA which also serves as calibrator (positive control=PC) and water (to prepare the reactions as well as a negative control=NC) are supplied in the MammaTyper® kit. Each patient sample/control is analyzed with each assay mix (1, 2 and 3). The analysis is performed in triplicate resulting in 3×3=9 reactions per sample/control. Assay-mix 1 contains the assays for the biomarkers ERBB2 (FAM) and ESR1 (JOE), assay mix 2 contains the biomarker assay MKI67 (FAM) and reference assay B2M (JOE) and assay mix 3 contains the biomarker assay PGR (FAM) and reference assay CALM2 (JOE). The two reference assays are used to determine whether sufficient analyte (RNA) is present for an analysis of the patient sample. Invalid samples must not be used for calculation of results. For valid samples (sufficient RNA) the analysis starts with the calculation of the combined reference (CombRefSample, geometric mean value of the median Cq values of B2M and CALM2). The marker specific ΔCqSample value is then determined by subtraction of CombRefSample from the four median Cq values of the biomarkers ERBB2, ESR1, PGR and MKI67.

The resulting marker-specific ΔCq values are then corrected using the calibrator, by subtracting a calibrator ΔCqPC. The CombRefPC (CombRefPC, geometric mean value of the median Cq values of B2M and CALM2 of the positive control, PC) is subtracted from the respective marker Cq value of the positive control, to calculate the marker-specific calibrator.

This results in the ΔΔCq value:

ΔΔCq=ΔCqSample−ΔCqPC,

with

ΔCqSample=(Median Cq[MarkerSample]−[CombRefSample]),

and

ΔCqPC=(Median Cq[MarkerPC]−[CombRefPC]).

The final results (40-ΔΔCq values) are obtained by subtracting the ΔΔCq values from the total number of PCR cycles (40), so that test results are positively correlated with marker expression, a format that facilitates interpretation for clinical decision making.

For tumor subtyping, the marker-specific 40-ΔΔCq values are dichotomized into “positive” or “negative” based on a clinically validated threshold value (cut-off). In addition, continuous values of each quantitative marker determination are reported. The combination of the four marker results (pos/neg) can then be used to determine the molecular subtype of the tumor sample (Table 1). For determination of a subtype, it is, therefore, necessary to analyze all three assay mixes in one run to obtain the four 40-ΔΔCq values of the sample.

TABLE 1
Translation of MammaTyper ® single marker results
into molecular subtypes according to the 13^{th}
St Gallen guidelines (Goldhirsch A. et al., 2013).

| ERBB2 | ESR1 | PGR | MKI67 | St Gallen Subtype |
|---|---|---|---|---|
| pos | pos | pos | pos | Luminal B-like (HER2 positive) |
| pos | pos | pos | neg | Luminal B-like (HER2 positive) |
| pos | pos | neg | pos | Luminal B-like (HER2 positive) |
| pos | pos | neg | neg | Luminal B-like (HER2 positive) |
| pos | neg | pos | pos | Not defined |
| pos | neg | pos | neg | Not defined |
| pos | neg | neg | pos | HER2 positive (non-luminal) |
| pos | neg | neg | neg | HER2 positive (non-luminal) |
| neg | pos | pos | pos | Luminal B-like (HER2 negative) |
| neg | pos | pos | neg | Luminal A-like |
| neg | pos | neg | pos | Luminal B-like (HER2 negative) |
| neg | pos | neg | neg | Luminal B-like (HER2 negative) |
| neg | neg | pos | pos | Not defined |
| neg | neg | pos | neg | Not defined |
| neg | neg | neg | pos | Triple-negative (ductal) |
| neg | neg | neg | neg | Triple-negative (ductal) |

### Example 3: Training and Validation of Unsealed Scores

Total RNA was extracted from whole surface 10 μm sections from FFPE breast cancer samples with a known RS result and a tumor cell content ≥20%. ERBB2, ESR1, PGR and MKI67 mRNA expression was measured by RT-qPCR on a CFX96 qPCR cycler using the MammaTyper® kit. On MammaTyper ERBB2-negative samples (40-□□Cq<40.4) and ESR1-positive samples (40-□□Cq≥36.9) a prediction model for an RS≤25 result was established using multivariable logistic regression. Based on this model and the training data, two cut-offs for confident prediction of low chemotherapy benefit patients in a clinical setting were established at 95% and 97.5% specificity. The model and the cut-offs were then fixed and validated in a second, separate set of breast cancer samples. ROC analysis was used to characterize predictive power of the continuous values resulting from the prediction model. Positive and negative predictive values for detection of an RS≤25 result were also determined on the validation samples using the two pre-defined cut-offs.

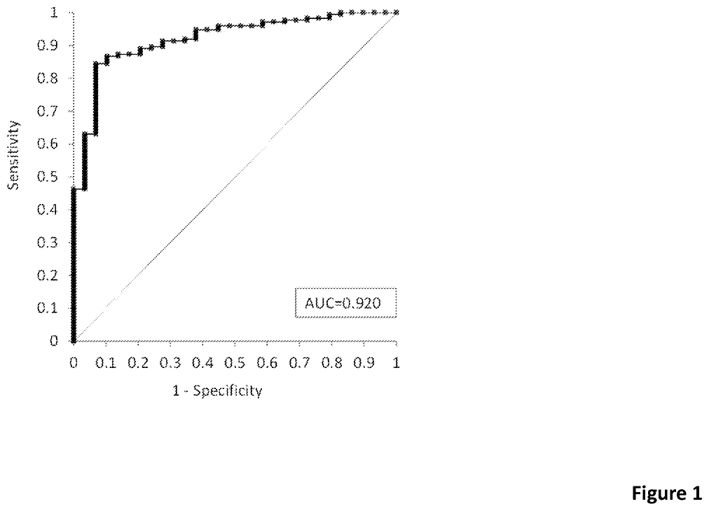

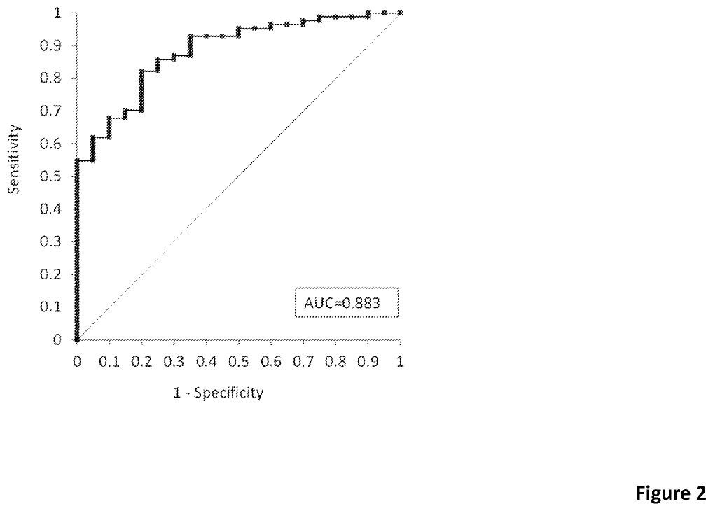

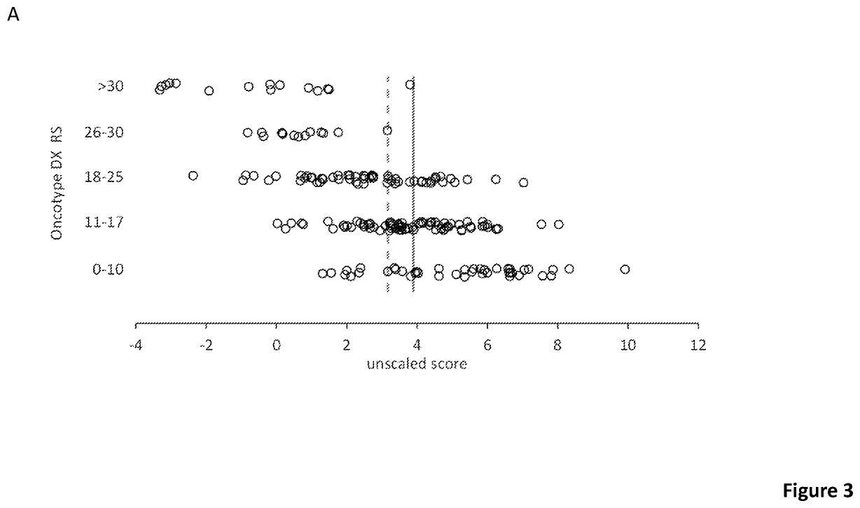

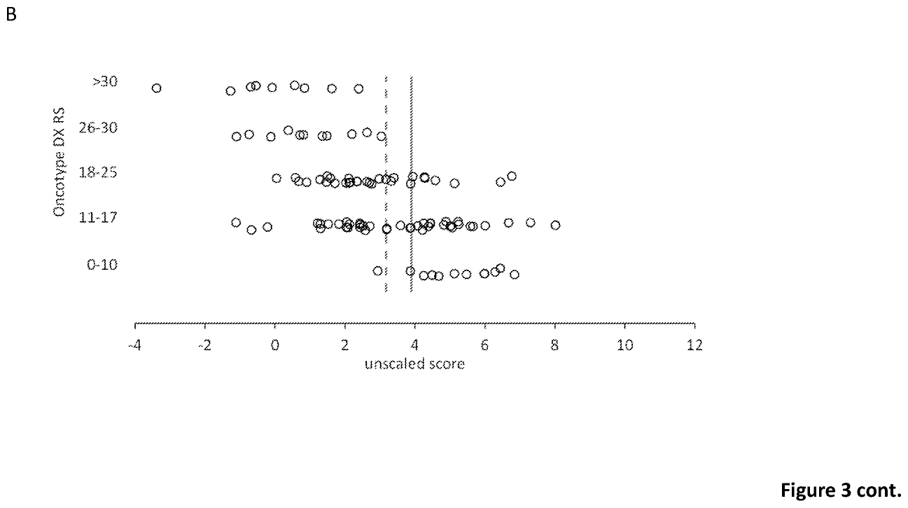

The sample set for training of the prediction model encompassed 202 samples, including 29 samples (14.4%) with an RS>25. In an initial multivariable model with all four markers, PGR and MKI67 were the strongest predictors while the influence of ESR1 in the model was lower, but still significant. ERBB2 was no significant predictor in this set of ERBB2-negative samples and was therefore excluded from the final model which was based on three markers only. This three marker model achieved an AUC of 0.920 (95% CI: 0.871-0.968) (FIG. 1) in the training samples. When applying the fixed model from the training dataset to a second separately collected set of 104 samples containing 20 samples (19.2%) with an RS>25, an AUC of 0.883 (95% CI: 0.810-0.955) was documented (FIG. 2). When further applying the two predefined cut-offs established in the training set, 45 and 36 of the 104 validation samples (43.3% and 34.6%) (FIG. 3) had a predicted low chemotherapy benefit result (RS≤25). Even with the less stringent cut-off, not a single one of the RS>25 cases from the validation cohort was falsely predicted as RS≤25 sample.

Based on the above, two unscaled scores (su) for predicting an Oncotype DX′ low risk RS (RS≤25) were established using multivariable logistic regression. The unscaled scores were (su=score unscaled; REL(ESR1), REL(PGR), REL(MKI67)=relative expression levels as determined with the MammaTyper® kit in 40-ΔΔCq):

su=12.313+0.539·REL(ESR1)+0.902·REL(PGR)−1.602·REL(MKI67)(=“score 1”), and

su=25.490+0.847·REL(PGR)−1.353·REL(MKI67)(=“score 2”).

REL(MKI67) was determined using CALM2 as the only reference gene.

Higher unscaled score values are associated with higher probabilities of an RS≤25 result. Samples with high probability of RS≤25 can be separated from samples with low probability of RS≤25 using a cut-off of, for example, 3.892 (high probability of RS≤25 when score ≥3.892, low probability of RS≤25 when score<3.892).

The signs (+/−) can be exchanged in the respective formula, which yields an unscaled score correlated with the probability of an RS>25 (high risk) result rather than an RS≤25 (low risk) result.

In addition to the binary classification using a cut-off, for each sample the individual predicted probability of pCR can be calculated.

$$Predicted probability of RS \leq 25 q = \frac{exp ( s u )}{( 1 + exp ( s u ) )}$$

The unscaled scores were trained on the data derived from a CFX96 qPCR instrument. To apply the score on data derived from a qPCR platform other than CFX96, the 40-ΔΔCq values derived from such platform can be transformed into 40-ΔΔCq values as expected for this sample on the CFX96 system. This transformation of 40-ΔΔCq values can be done by using a linear equation, or by adding/subtracting a pre-defined Cq value to/from the respective 40-ΔΔCq values, thereby simulating CFX96 expression values. Another possible approach is to transfer the score to the other platform. In a dataset in which the same samples were measured with the CFX96 system and the other platform, the 40-ΔΔCq values from the other platform can be used as predictors to determine the pCR score which is calculated from the 40-ΔΔCq values determined on the CFX96 system for the same samples using linear regression analysis.

### Example 4: Further Validation of Prediction Score

The algorithm and the main cut-off established based on the finding cohort (see Example 3) were then applied to another set of separately collected samples for which RS values had been previously determined.

The prediction algorithm (score 1) was also applied to a set of samples measured on the CFX96 system for which simulated RS values were available. These RS simulations were based on Nanostring measurements of all targets and reference genes from Oncotype DX and calculation of RS values based on an algorithm which had also been trained against real RS values (Bayani et al., 2017).

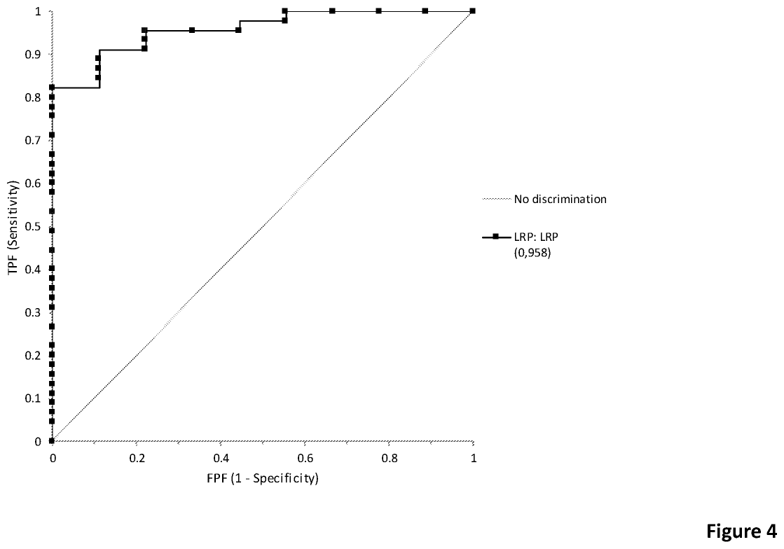

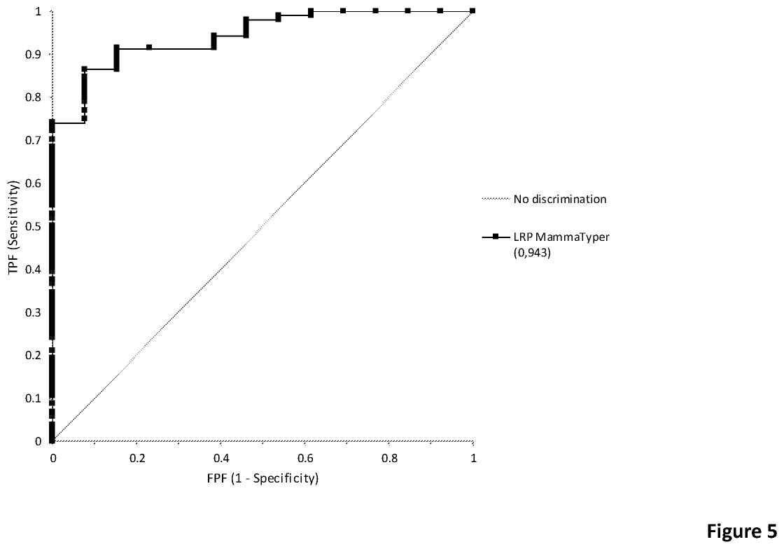

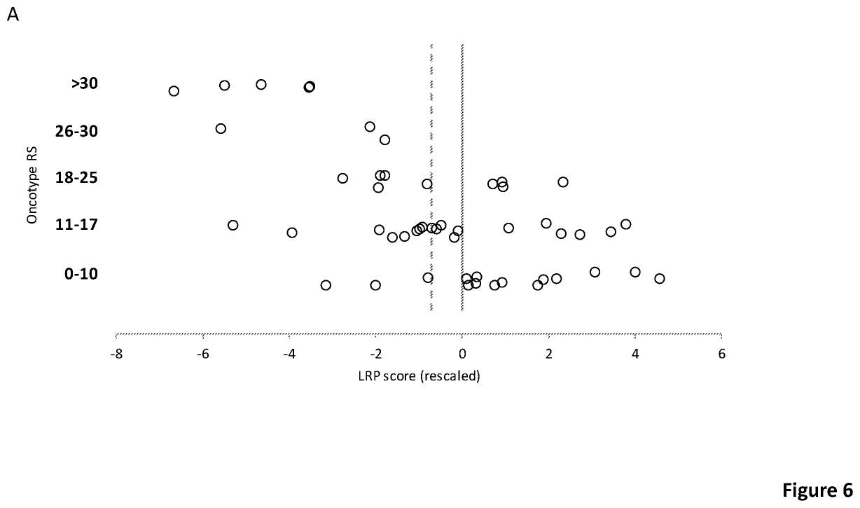

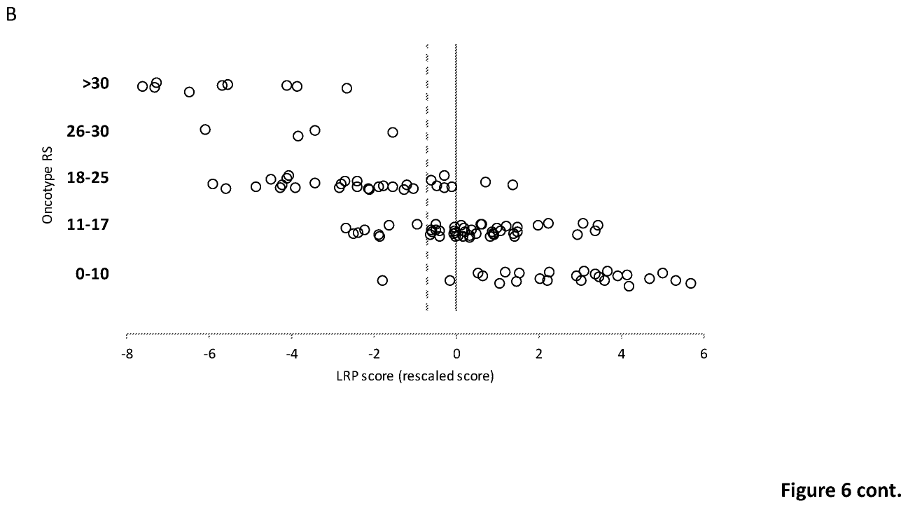

The final rescaled score, which is also referred to as low-risk-predict (LPR) score and which was obtained by subtracting the main cut-off (3.892) from the unscaled score su (here: score 1), achieved high AUCs in both cohorts (FIG. 4 and FIG. 5). When applying the more stringent cut-off of 0 but also when applying the less stringent cut-off of −0.722 no (simulated) RS value >25 was missed (FIG. 6).

### REFERENCES

Goldhirsch A, Winer E P, Coates A S, et al. Personalizing the treatment of women with early breast cancer: highlights of the St Gallen International Expert Consensus on the Primary Therapy of Early Breast Cancer 2013. Ann Oncol 2013; 24:2206-2223.
Yerushalmi R, Woods R, Ravdin P M, et al. Ki67 in breast cancer: Prognostic and predictive potential. The Lancet Oncology 2010; 11:174-183.
Varga Z, Diebold J, Dommann-Scherrer C, et al. How reliable is Ki-67 immunohistochemistry in grade 2 breast carcinomas? A QA study of the Swiss Working Group of Breast- and Gynecopathologists. PLoS One 2012; 7:e37379.
Polley M C, Leung S C Y, McShane L M, et al. An international Ki67 reproducibility study. J Natl Cancer Inst 2013; 105:1897-1906.
Levine M N, Julian J A, Bedard P L, et al. Prospective Evaluation of the 21-Gene Recurrence Score Assay for Breast Cancer Decision-Making in Ontario. J Clin Oncol 2016; 34:1065-1071.
Martin M, Gonzalez-Rivera M, Morales S, et al. Prospective study of the impact of the Prosigna assay on adjuvant clinical decision-making in unselected patients with estrogen receptor positive, human epidermal growth factor receptor negative, node negative early-stage breast cancer. Curr Med Res Opin 2015; 31:1129-1137.
Harris L N, Ismaila N, McShane L M, et al. Use of Biomarkers to Guide Decisions on Adjuvant Systemic Therapy for Women With Early-Stage Invasive Breast Cancer: American Society of Clinical Oncology Clinical Practice Guideline. J Clin Oncol 2016; 34:1134-1150.
Sparano J A, Gray R J, Della Makower F, et al. Prospective Validation of a 21-Gene Expression Assay in Breast Cancer. N Engl J Med 2015; 373:2005-2014.
Flanagan M B, Dabbs D J, Brufsky A M, et al. Histopathologic variables predict Oncotype DX recurrence score. Mod Pathol 2008; 21:1255-1261.
Klein M E, Dabbs D J, Shuai Y, et al. Prediction of the Oncotype DX recurrence score: use of pathology-generated equations derived by linear regression analysis. Mod Pathol 2013; 26:658-664.
Ingoldsby H, Webber M, Wall D, et al. Prediction of Oncotype DX and TAILORx risk categories using histopathological and immunohistochemical markers by classification and regression tree (CART) analysis. Breast 2013; 22:879-886.
Sahebj am S, Aloyz R, Pilavdzic D, et al. Ki 67 is a major, but not the sole determinant of Oncotype Dx recurrence score. Br J Cancer 2011; 105:1342-1345.
Turner B M, Skinner K A, Tang P, et al. Use of modified Magee equations and histologic criteria to predict the Oncotype DX recurrence score. Mod Pathol 2015; 28:921-931.
Harowicz M R, Robinson T J, Dinan M A, et al. Algorithms for prediction of the Oncotype DX recurrence score using clinicopathologic data: a review and comparison using an independent dataset. Breast Cancer Res Treat 2017; 162:1-10.
Kim H, Umbricht C B, Illei P B, et al. Optimizing the Use of Gene Expression Profiling in Early-Stage Breast Cancer. J Clin Oncol 2016; 34:4390-4397.
Bartlett J M S, Christiansen J, Gustayson M, et al. Validation of the IHC4 Breast Cancer Prognostic Algorithm Using Multiple Approaches on the Multinational TEAM Clinical Trial. Arch Pathol Lab Med 2016; 140:66-74.
Sparano J A, et al. Adjuvant Chemotherapy Guided by a 21-Gene Expression Assay in Breast Cancer. N Engl J Med 2018; 379(2):111-121.
Varga Z, Lebeau A, Bu H, et al. An international reproducibility study validating quantitative determination of ERBB2, ESR1, PGR, and MKI67 mRNA in breast cancer using MammaTyper®. Breast Cancer Res 2017; 19:55.
Laible M, Schlombs K, Kaiser K, et al. Technical validation of an RT-qPCR in vitro diagnostic test system for the determination of breast cancer molecular subtypes by quantification of ERBB2, ESR1, PGR and MKI67 mRNA levels from formalin-fixed paraffin-embedded breast tumor specimens. BMC Cancer 2016; 16:398.
Bayani J, Yao C Q, Quintayo M A, et al. Molecular stratification of early breast cancer identifies drug targets to drive stratified medicine. NPJ Breast Cancer. 2017; 3:3.

1. Method of predicting the probability of an Oncotype DX® low risk recurrence score (RS) result (RS≤25) for an ERBB2-negative breast cancer patient, said method comprising:
calculating a score unscaled (su) based on the relative expression levels of mRNA of ESR1, PGR and MKI67 in a breast tumor sample of the breast cancer patient as determined by reverse transcription quantitative PCR (RT-qPCR), wherein
a) a higher score su indicates a higher probability of RS≤25, wherein a higher relative expression level of mRNA of ESR1 is associated with a higher su, a higher relative expression level of mRNA of PGR is associated with a higher su, and a higher relative expression level of mRNA of MKI67 is associated with a lower su; or
b) a lower score su indicates a higher probability of RS≤25, wherein a higher relative expression level of mRNA of ESR1 is associated with a lower su, a higher relative expression level of mRNA of PGR is associated with a lower su, and a higher relative expression level of mRNA of MKI67 is associated with a higher su.

2. The method according to claim 1, wherein the method comprises, prior to calculating su:
determining the relative expression levels of mRNA of ESR1, PGR and MKI67 in the breast tumor sample by RT-qPCR.

3. The method according to claim 1 or 2, wherein the breast cancer is an ERBB2-negative and ESR1-positive breast cancer.

4. The method according to any one of claims 1 to 3, wherein, in the calculation of su, the relative expression levels (RELs) of mRNA of ESR1, PGR and MKI67 are weighted as follows:
REL(ESR1):REL(PGR):REL(MKI67)=0.60(±0.09):1(±0.15):1.78(±0.27).

5. The method according to claim 4, wherein a higher score su indicates a higher probability of RS≤25, and wherein su is calculated by using the formula:
su=BASELINE+WF(ESR1)·REL(ESR1)+WF(PGR)·REL(PGR)−WF(MKI67)·REL(MKI67),
wherein WF(ESR1) is a weighting factor for REL(ESR1), WF(PGR) is a weighting factor for REL(PGR), and WF(MKI67) is a weighting factor for REL(MKI67).

6. The method according to any one of claims 1 to 5, wherein a higher score su indicates a higher probability of RS≤25, and wherein su is calculated by using the formula:
su=12.313+0.539·REL(ESR1)+0.902·REL(PGR)−1.602·REL(MKI67).

7. The method according to claim 4, wherein a lower score su indicates a higher probability of RS≤25, and wherein su is calculated by using the formula:
su=−BASELINE−WF(ESR1)·REL(ESR1)−WF(PGR)·REL(PGR)+WF(MKI67)·REL(MKI67),
wherein WF(ESR1) is a weighting factor for REL(ESR1), WF(PGR) is a weighting factor for REL(PGR), and WF(MKI67) is a weighting factor for REL(MKI67).

8. The method according to any one of claims 1 to 4 and 7, wherein a lower score su indicates a higher probability of RS≤25, and wherein su is calculated by using the formula:
su=−12.313−0.539·REL(ESR1)−0.902·REL(PGR)+1.602·REL(MKI67).

9. The method according to any one of claims 1 to 8, further comprising:
calculating a predicted probability of RS≤25 q, wherein
a) if a higher score su indicates a higher probability of RS≤2.5, q is calculated by using the formula
$$q = \frac{exp ( s u )}{( 1 + exp ( s u ) )} ;$$
and
b) if a lower score su indicates a higher probability of RS≤25, q is calculated by using the formula
$$q = 1 - \frac{exp ( s u )}{( 1 + exp ( s u ) )} ,$$
wherein, preferably, a q which is equal to or greater than a pre-defined threshold indicates a high probability of RS≤25, and a q which is lower than a pre-defined threshold indicates a low probability of RS≤25.

10. The method according to any one of claims 1 to 8, further comprising:
calculating a clinical score s based on su, wherein s has a scale from 0 to 100 or from −10 to 10.

11. The method according to any one of claims 1 to 8 and 10, wherein
a) if a higher score su indicates a higher probability of RS≤25, a score s or a score su which is equal to or greater than a pre-defined threshold indicates a high probability of RS≤25, and a score s or a score su which is lower than the pre-defined threshold indicates a low probability of RS≤25; and
b) if a lower score su indicates a higher probability of RS≤25, a score s or a score su which is lower than a pre-defined threshold indicates a high probability of RS≤25, and a score s or a score su which is equal to or greater than the pre-defined threshold indicates a low probability of RS≤25.

12. Method of predicting the probability of an Oncotype DX® low risk recurrence score (RS) result (RS≤25) for an ERBB2-negative breast cancer patient, said method comprising:
calculating a score unscaled (su) based on the relative expression levels of mRNA of PGR and MKI67 in a breast tumor sample of the breast cancer patient as determined by reverse transcription quantitative PCR (RT-qPCR), wherein
a) a higher score su indicates a higher probability of RS≤25, wherein a higher relative expression level of mRNA of PGR is associated with a higher su, and a higher relative expression level of mRNA of MKI67 is associated with a lower su; or
b) a lower score su indicates a higher probability of RS≤25, wherein a higher relative expression level of mRNA of PGR is associated with a lower su, and a higher relative expression level of mRNA of MKI67 is associated with a higher su.

13. The method according to claim 12, wherein the method comprises, prior to calculating su:
determining the relative expression levels of mRNA of PGR and MKI67 in the breast tumor sample by RT-qPCR.

14. The method according to claim 12 or 13, wherein the breast cancer is an ERBB2-negative and ESR1-positive breast cancer.

15. The method according to any one of claims 12 to 14, wherein, in the calculation of su, the relative expression levels (RELs) of mRNA of PGR and MKI67 are weighted as follows:
REL(PGR):REL(MKI67)=1(±0.15):1.60(±0.24).

16. The method according to claim 15, wherein a higher score su indicates a higher probability of RS≤25, and wherein su is calculated by using the formula:
su=BASELINE+WF(PGR)·REL(PGR)−WF(MKI67)·REL(MKI67),
wherein WF(PGR) is a weighting factor for REL(PGR), and WF(MKI67) is a weighting factor for REL(MKI67).

17. The method according to any one of claims 12 to 16, wherein a higher score su indicates a higher probability of RS≤25, and wherein su is calculated by using the formula:
su=25.490+0.847·REL(PGR)−1.353·REL(MKI67).

18. The method according to claim 15, wherein a lower score su indicates a higher probability of RS≤25, and wherein su is calculated by using the formula:
su=−BASELINE−WF(PGR)·REL(PGR)+WF(MKI67)·REL(MKI67),
wherein WF(PGR) is a weighting factor for REL(PGR), and WF(MKI67) is a weighting factor for REL(MKI67).

19. The method according to any one of claims 12 to 15 and 18, wherein a lower score su indicates a higher probability of RS≤25, and wherein su is calculated by using the formula:
su=−25.490−0.847·REL(PGR)+1.353·REL(MKI67).

20. The method according to any one of claims 12 to 19, further comprising:
calculating a predicted probability of RS≤25 q, wherein
a) if a higher score su indicates a higher probability of RS≤25, q is calculated by using the formula
$$q = \frac{exp ( s u )}{( 1 + exp ( s u ) )} ;$$
and
b) if a lower score su indicates a higher probability of RS≤25, q is calculated by using the formula
$$q = 1 - \frac{exp ( s u )}{( 1 + exp ( s u ) )} ,$$
wherein, preferably, a q which is equal to or greater than a pre-defined threshold indicates a high probability of RS≤25, and a q which is lower than a pre-defined threshold indicates a low probability of RS≤25.

21. The method according to any one of claims 12 to 19, further comprising:
calculating a clinical score s based on su, wherein s has a scale from 0 to 100 or from −10 to 10.

22. The method according to any one of claims 12 to 19 and 21, wherein
a) if a higher score su indicates a higher probability of RS≤25, a score s or a score su which is equal to or greater than a pre-defined threshold indicates a high probability of RS≤25, and a score s or a score su which is lower than the pre-defined threshold indicates a low probability of RS≤25; and
b) if a lower score su indicates a higher probability of RS≤25, a score s or a score su which is lower than a pre-defined threshold indicates a high probability of RS≤25, and a score s or a score su which is equal to or greater than the pre-defined threshold indicates a low probability of RS≤25.

23. Method of predicting the probability of an Oncotype DX® low risk recurrence score (RS) result (RS≤25) for an ERBB2-negative breast cancer patient, said method comprising:
calculating a score unscaled (su) based on the relative expression levels of mRNA of ERBB2, ESR1, PGR and MKI67 in a breast tumor sample of the breast cancer patient as determined by reverse transcription quantitative PCR (RT-qPCR), wherein
a) a higher score su indicates a higher probability of RS≤25, wherein a higher relative expression level of mRNA of ERBB2 is associated with a lower su, a higher relative expression level of mRNA of ESR1 is associated with a higher su, a higher relative expression level of mRNA of PGR is associated with a higher su, and a higher relative expression level of mRNA of MKI67 is associated with a lower su; or
b) a lower score su indicates a higher probability of RS≤25, wherein a higher relative expression level of mRNA of ERBB2 is associated with a higher su, a higher relative expression level of mRNA of ESR1 is associated with a lower su, a higher relative expression level of mRNA of PGR is associated with a lower su, and a higher relative expression level of mRNA of MKI67 is associated with a higher su.

24. The method according to claim 23, wherein the method comprises, prior to calculating su:
determining the relative expression levels of mRNA of ERBB2, ESR1, PGR and MKI67 in the breast tumor sample by RT-qPCR.

25. Method for selecting a breast cancer treatment for an ERBB2-negative breast cancer patient, said method comprising:
predicting the probability of an Oncotype DX® low risk recurrence score (RS) result (RS≤25) for the breast cancer patient by using a method according to any one of claims 1 to 24; and
selecting endocrine therapy as the breast cancer treatment for the breast cancer patient if a high probability of RS≤25 is predicted.

26. The method according to claim 25, wherein a high probability of RS≤25 is predicted if su, q or s is higher than a pre-defined threshold.

27. Method of treatment of ERBB2-negative breast cancer in a breast cancer patient comprising:
selecting a breast cancer treatment for the breast cancer patient by using a method according to claim 25 or 26; and
administering the selected breast cancer treatment to the breast cancer patient.

28. Use of a kit in a method according to any one of claims 2, 13 and 24, wherein the kit comprises:
at least one pair of ERBB2-specific primers;
at least one pair of ESR1-specific primers;
at least one pair of PGR-specific primers; and/or
at least one pair of MKI67-specific primers.

29. The use according to claim 28, wherein the kit further comprises at least one ERBB2-specific probe, at least one ESR1-specific probe, at least one PGR-specific probe and/or at least one MKI67-specific probe.

30. The use according to claim 28 or 29, wherein the kit further comprises at least one pair of reference gene-specific primers and, optionally, at least one reference gene-specific probe.

31. The use according to any one of claims 28 to 30, wherein the reference gene is selected from the group consisting of B2M, CALM2, TBP, PUM1, MRLP19, GUSB, RPL37A and CYFIP1.
